(19) United States
(12) Patent Application Publication
(10) Pub. No.: US 2007/0082028 A1
(43) Pub. Date: Apr. 12, 2007

(54) Compositions and methods for inducing satiety and reducing caloric intake

(76) Inventors: William Ronald Aimutis JR., Blaine, MN (US); Eugene Terry Finocchiaro, West Amwell, NJ (US); Teresa Marie Paeschke, Minneapolis, MN (US); Thomas E. Sox, Ambler, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/245,910

(22) Filed: Oct. 7, 2005

(51) Int. Cl. A61K 47/00; A61K 33/42

(52) U.S. Cl. 424/439; 424/602

(57) ABSTRACT

Compositions and methods for inducing statiety and for reducing caloric intake having at least soluble anionic fiber and at least one soluble cation. The compositions may be solid, fluid, or mixtures thereof.

### CROSS-REFERENCE TO RELATED APPLICATIONS

This case is related to U.S. patent application Ser. No. ______, entitled “COMPOSITIONS AND METHODS FOR REDUCING FOOD INTAKE AND CONTROLLING WEIGHT” (docket number MSP5038); U.S. patent application Ser. No ______, entitled “METHODS FOR REDUCING CALORIE INTAKE” (docket number MSP5039), U.S. patent application Ser. No. ______, entitled “METHODS FOR ACHIEVING AND MAINTAINING WEIGHT LOSS” (docket number MSP5041); U.S. patent application Ser. No. ______, entitled “METHODS FOR REDUCING WEIGHT” (docket number MSP5042); U.S. patent application Ser. No. ______, entitled “COMPOSITIONS AND METHODS FOR REDUCING FOOD INTAKE AND CONTROLLING WEIGHT” (docket number MSP5043); U.S. patent application Ser. No. ______, entitled “COMPOSITIONS AND METHODS FOR REDUCING FOOD INTAKE AND CONTROLLING WEIGHT” (docket number MSP5044); U.S. patent application Ser. No. ______, entitled “METHODS FOR WEIGHT MANAGEMENT” (docket number MSP5045); U.S. patent application Ser. No. ______, entitled “METHODS FOR INDUCING SATIETY, REDUCING FOOD INTAKE AND REDUCING WEIGHT” (docket number MSP5046); U.S. patent application Ser. No. ______, entitled “COMPOSITIONS AND METHODS FOR REDUCING FOOD INTAKE AND CONTROLLING WEIGHT (docket number MSP5047); U.S. patent application Ser. No. ______, entitled “FIBER SATIETY COMPOSITIONS” (docket number 10790-056001); and U.S. patent application Ser. No. ______, entitled “FIBER SATIETY COMPOSITIONS” (docket number 10790-056002), each filed concurrently herewith on Oct. 7, 2005.

### FIELD OF THE INVENTION

The present invention is directed to a ingestible compositions that include at least one soluble anionic fiber and at least one soluble cation source, and methods of using the ingestible compositions to induce satiety and to decrease caloric intake.

### BACKGROUND OF THE INVENTION

Diabetes and obesity are common ailments in the United States and other Western cultures. A study by researchers at RTI International and the Centers for Disease Control estimated that U.S. obesity-attributable medical expenditures reached $75 billion in 2003. Obesity has been shown to promote many chronic diseases, including type 2 diabetes, cardiovascular disease, several types of cancer, and gallbladder disease.

Adequate dietary intake of soluble fiber has been associated with a number of health benefits, including decreased blood cholesterol levels, improved glycemic control, and the induction of satiety and satiation in individuals. Consumers have been resistant to increasing soluble fiber amounts in their diet, however, often due to the negative organoleptic characteristics, such as, sliminess, excessive viscosity, excessive dryness and poor flavor, that are associated with food products that include soluble fiber.

What is needed is a stable, organoleptically acceptable product that delivers a calcium salt and a greater than normal level of alginate while preventing these two components from reacting during its shelf life and prior to ingesting the product. The product can be a cookie or mini bar with a fruit component.

### SUMMARY OF THE INVENTION

The present invention solves the above needs by providing an ingestible composition comprising, consisting of and/or consisting essentially of at least one soluble anionic fiber and at least one soluble cation source, wherein the ingestible composition has an SE of between about 100% and about 250%.

Another embodiment of the present invention is directed to an ingestible composition comprising, consisting of, and/or consisting essentially of solid phase comprising, consisting of, and/or consisting essentially of at least one soluble anionic fiber in a total amount of from about 0.5 g to about 10 g per serving and a fluid phase in intimate contact with the solid phase, the fluid phase comprising, consisting of, and/or consisting essentially of a soluble calcium source in an amount of from about 50 to about 300 mg of elemental calcium per serving, wherein the ingestible composition has an SE of between about 100% and about 250%.

A further embodiment of the present invention is directed to a method for inducing satiety in an animal, the method comprising, consisting of and/or consisting essentially of the step of orally administering to the animal a serving of a preload composition, the preload composition comprising, consisting of and/or consisting essentially at least one soluble anionic fiber and at least one soluble cation source, wherein the preload composition has an SE of between about 100% and about 250%.

Yet another embodiment of the present invention is a method for inducing satiety in an animal, the method comprising, consisting of and/or consisting essentially of the step of orally administering to the animal a serving of a preload composition, the preload comprising, consisting of and/or consisting essentially of a solid phase comprising, consisting of and/or consisting essentially of at least one soluble anionic fiber in a total amount of from about 0.5 g to about 10 g per serving and a fluid phase in intimate contact with the solid phase, the fluid phase comprising, consisting of and/or consisting essentially of a soluble calcium source in an amount of from about 50 to about 500 mg of elemental calcium per serving, wherein the preload has an SE of between about 100% and about 250%.

A still further embodiment of the present invention is a method for reducing caloric intake in an animal, the method comprising, consisting of and/or consisting essentially of orally administering to the animal at least one serving of a preload composition, the preload composition comprising, consisting of and/or consisting essentially of at least one soluble anionic fibers and at least one soluble cation source, wherein the preload has an SE of between about 100% and about 250%.

An additional embodiment of the present invention is method for reducing caloric intake in an animal, the method comprising, consisting of and/or consisting essentially of the step of orally administering to the animal a serving of a preload composition, the preload composition comprising, consisting of and/or consisting essentially of a solid phase comprising, consisting of and/or consisting essentially of at least one soluble anionic fiber in a total amount of from about 0.5 g to about 10 g per serving and a fluid phase in intimate contact with the solid phase, the fluid phase comprising, consisting of and/or consisting essentially of a soluble calcium source in an amount of from about 50 to about 300 mg of elemental calcium per serving, wherein the preload composition has an SE of between about 100% and about 250%.

A further embodiment of the present invention is directed to a method for reducing weight in an animal, the method comprising, consisting of and/or consisting essentially of the step of orally administering to the animal a serving of an ingestible composition comprising, consisting of and/or consisting essentially of an extruded food product, wherein the extruded food product comprises, consists of and/or consists essentially of at least one soluble anionic fiber and at least one soluble cation source.

Another embodiment of the present invention is directed to a method for reducing weight in an animal, the method comprising, consisting of and/or consisting essentially of the step of orally administering to the animal a serving of an ingestible composition comprising, consisting of and/or consisting essentially of a solid phase comprising, consisting of and/or consisting essentially of at least one soluble anionic fiber in a total amount of from about 0.5 g to about 10 g per serving and a fluid phase in intimate contact with the solid phase, the fluid phase comprising, consisting of and/or consisting essentially of a soluble calcium source in an amount in an amount to provide from about 50 to about 300 mg of elemental calcium per serving.

Yet another embodiment of the present invention is directed to an ingestible composition comprising, consisting of and/or consisting essentially of a solid phase comprising, consisting of and/or consisting essentially of ate least one soluble anionic fiber in a total amount of from about 0.5 g to about 10 g per serving and a fluid phase in intimate contact with the solid phase, the fluid phase comprising, consisting of and/or consisting essentially of a soluble calcium source in an amount to provide from about 50 to about 300 mg of elemental calcium per serving, wherein ingestible composition has an SE of between about 100% and about 250%.

### BRIEF DESCRIPTION OF DRAWINGS

FIG. 1 is a graph depicting the effects of an embodiment of the present invention on intestinal viscosity.

### DETAILED DESCRIPTION OF THE INVENTION

As used herein, unless indicated otherwise, the terms “alginate,” “pectin,” “carrageenan,” “polygeenan,” or “gellan” refers to all forms (e.g., protonated or salt forms, such as sodium, potassium, and ammonium salt forms and having varying average molecular weight ranges) of the soluble anionic fiber type.

As used herein, unless indicated otherwise, the term “alginic acid” includes not only the material in protonated form but also the related salts of alginate, including but not limited to sodium, potassium, and ammonium alginate.

As used herein, unless indicated otherwise, the term “protected” means that the source has been treated in such a way, as illustrated below, to delay (e.g., until during or after ingestion or until a certain pH range has been reached) reaction of the at least one multivalent cation with the soluble anionic fiber as compared to an unprotected multivalent cation.

As used herein, the term SE or Satiety Efficiency Index means, unless otherwise defined, caloric reduction in a given meal due to preload divided by the caloric value of the preload. For example, if a person consumes a 1000 calorie lunch without ingesting a preload, but consumes a 900 calorie lunch after ingesting a 200 calorie preload, the preload would have 0.5 or 50% SE. Another example is a person consumes a 1000 calorie lunch without ingesting a preload, but consumes a 800 calorie lunch after ingesting a 100 calorie preload, the preload would have a 2.0 or 200% SE. As can be seen, the greater the SE, the greater the effect of the preload on the next meal.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, a recitation of a range of values is merely intended to serve as a shorthand method of referring individually to each separate value falling within the range, and each separate value is incorporated into the specification as if it were individually recited herein.

The compositions of this invention reduce caloric intake and induce satiety. The inventors believe that this arises from the enhanced viscosity produced by the interactions of soluble cation source and at least one soluble anionic fiber.

Soluble Anionic Fiber

Any soluble anionic fiber should be acceptable for the purposes of this invention. Suitable soluble anionic fibers include algniate, pectin, gellan, soluble fibers that contain carboxylate substituents, carrageenan, polygeenan, and marine algae-derived polymers that contain sulfate substituents.

Also included within the scope of soluble anionic fibers are other plant derived and synthetic or semisynthetic polymers that contain sufficient carboxylate, sulfate, or other anionic moieties to undergo gelling in the presence of sufficient levels of multivalent cation.

At least one source of soluble anionic fiber may be used in these compositions, and the at least one source of soluble anionic fiber may be combined with at least one source of soluble fiber that is uncharged at neutral pH. Thus, in certain cases, two or more soluble anionic fibers types are included, such as, alginate and pectin, alginate and gellan, or pectin and gellan. In other cases, only one type of soluble anionic fiber is used, such as only alginate, only pectin, only carrageenan, or only gellan.

Soluble anionic fibers are commercially available, e.g., from ISP (Wayne, N.J.), TIC Gums, and CP Kelco.

An alginate can be a high guluronic acid alginate. For example, in certain cases, an alginate can exhibit a higher than 1:1 ratio of guluronic to mannuronic acids, such as in the range from about 1.2:1 to about 1.8:1, e.g., about 1.3:1, about 1.4:1, about 1.5:1, about 1.6:1, or about 1.7:1 or any value therebetween. Examples of high guluronic alginates (e.g., having a higher than 1:1 g:m ratios) include Manugel LBA, Manugel GHB, and Manugel DBP, which each have a g:m ratio of about 1.5.

While not being bound by theory, it is believed that high guluronic alginates can cross-link through multivalent cations, e.g., calcium ions, to form gels at the low pH regimes in the stomach. High guluronic alginates are also believed to electrostatically associate with pectins and/or gellans at low pHs, leading to gellation. In such cases, it may be useful to delay the introduction of multivalent cations until after formation of the mixed alginate/pectin or alginate/gellan gel, as multivalent cationic cross-links may stabilize the mixed gel after formation.

In other cases, an alginate can exhibit a ratio of guluronic to mannuronic acids (g:m ratio) of less than about 1:1, e.g., about 0.8:1 to about 0.4:1, such as about 0.5:1, about 0.6:1, or about 0.7:1 or any value therebetween. Keltone LV and Keltone HV are examples of high-mannuronic acids (e.g., having a g:m ratio of less than 1:1) having g:m ratios ranging from about 0.6:1 to about 0.7:1.

Methods for measuring the ratio of guluronic acids to mannuronic acids are known by those having ordinary skill in the art.

An alginate can exhibit any number average molecular weight range, such as a high molecular weight range (about 2.05×10⁵ to about 3×10⁵ Daltons or any value therebetween; examples include Manugel DPB, Keltone HV, and TIC 900 Alginate); a medium molecular weight range (about 1.38× to about 2×10⁵ Daltons or any value therebetween; examples include Manugel GHB); or a low molecular weight range (about 2× to about 1.35×10⁵ Daltons or any value therebetween; examples include Manugel LBA and Manugel LBB). Number average molecular weights can be determined by those having ordinary skill in the art, e.g., using size exclusion chromatography (SEC) combined with refractive index (R1) and multi-angle laser light scattering (MALLS).

In certain embodiments of an extruded food product, a low molecular weight alginate can be used (e.g., Manugel LBA), while in other cases a mixture of low molecular weight (e.g., Manugel LBA) and high molecular weight (e.g., Manugel DPB, Keltone HV) alginates can be used. In other cases, a mixture of low molecular weight (e.g., Manugel LBA) and medium molecular weight (e.g., Manugel GHB) alginates can be used. In yet other cases, one or more high molecular weight alginates can be used (e.g., Keltone HV, Manugel DPB).

A pectin can be a high-methoxy pectin (e.g., having greater than 50% esterified carboxylates), such as ISP HM70LV and CP Kelco USPL200. A pectin can exhibit any number average molecular weight range, including a low molecular weight range (about 1×10⁵ to about 1.20×10⁵ Daltons, e.g., CP Kelco USPL200), medium molecular weight range (about 1.25×10⁵ to about 1.45×10⁵, e.g., ISP HM70LV), or high molecular weight range (about 1.50×10⁵ to about 1.80×10⁵, e.g., TIC HM Pectin). In certain cases, a high-methoxy pectin can be obtained from pulp, e.g., as a by-product of orange juice processing.

A gellan soluble anionic fiber can also be used. Gellan fibers form strong gels at lower concentrations than alginates and/or pectins, and can cross-link with multivalent cation cations. For example, gellan can form gels with sodium, potassium, magnesium and calcium. Gellans for use in the invention include Kelcogel, available commercially from CP Kelco.

Fiber blends as described herein can also be used in the preparation of a solid ingestible composition like a formed food product where the fiber blend is a source of the soluble anionic fiber. A useful fiber blend can include an alginate soluble anionic fiber and a pectin soluble anionic fiber. A ratio of total alginate to total pectin in a blend can be from about 8:1 to about 5:1, or any value therebetween, such as about 7:1, about 6.5:1, about 6.2:1, or about 6.15:1. A ratio of a medium molecular weight alginate to a low molecular weight alginate can range from about 0.65:1 to about 2:1, or any value therebetween.

An alginate soluble anionic fiber in a blend can be a mixture of two or more alginate forms, e.g., a medium and low molecular weight alginate. In certain cases, a ratio of a medium molecular weight alginate to a low molecular weight alginate is about 0.8:1 to about 0.9:1. The high molecular weight alginate has been tested at about 0-2 g. The fiber blend combining low and medium molecular weight alginates with high methoxy pectin has been tested at about 0 to about 3 grams. The preferred range for both would be about 1 to about 2 grams.

The at least one soluble anionic fiber may be treated before, during, or after incorporation into an ingestible composition. For example, the at least one soluble anionic fiber can be processed, e.g., extruded, roll-dried, freeze-dried, dry blended, roll-blended, agglomerated, coated, or spray-dried.

For solid forms, a variety of extruded shapes of food products can be prepared by methods known to those having ordinary skill in the art, e.g., extruding, molding, pressing, wire cutting, and the like. For example, a single or double screw extruder can be used. Typically, a feeder meters in the raw ingredients to a barrel that includes the screw(s). The screw(s) conveys the raw material through the die that shapes the final product. Extrusion can take place under high temperatures and pressures or can be a non-cooking, forming process. Extruders are commercially available, e.g., from Buhler, Germany. Extrusion can be cold or hot extrusion.

Other processing methods are known to those having skilled in the art.

The amount of the at least one soluble anionic fiber included can vary, and will depend on the type of ingestible composition and the type of soluble anionic fiber used. For example, typically a solid ingestible composition will include from about 0.5 g to about 10 g total soluble anionic fiber per serving or any value therebetween. A preferred range of fiber intake in the compositions of this invention is about 0.25 g to about 5 g per serving, more preferably about 0.5 to about 3 g per serving, and most preferably about 1.0 to about 2.0 g per serving. In certain cases, an extruded food product can include an soluble anionic fiber at a total amount from about 22% to about 40% by weight of the extruded product or any value therebetween. In other cases, an extruded food product can include an soluble anionic fiber in a total amount of from about 4% to about 15% or any value therebetween, such as when only gellan is used. In yet other cases, an extruded food product can include an soluble anionic fiber at a total amount of from about 18% to about 25% by weight, for example, when combinations of gellan and alginate or gellan and pectin are used.

In addition to the at least one soluble anionic fiber, a solid ingestible composition can include ingredients that may be treated in a similar manner as the at least one soluble anionic fiber. For example, such ingredient can be co-extruded with the soluble anionic fiber, co-processed with the soluble anionic fiber, or co-spray-dried with the soluble anionic fiber. Such treatment can help to reduce sliminess of the ingestible composition in the mouth and to aid in hydration and gellation of the fibers in the stomach and/or small intestine. Without being bound by any theory, it is believed that co-treatment of the soluble anionic fiber(s) with such ingredient prevents early gellation and hydration of the fibers in the mouth, leading to sliminess and unpalatability. In addition, co-treatment may delay hydration and subsequent gellation of the soluble anionic fibers (either with other soluble anionic fibers or with multivalent cations) until the ingestible composition reaches the stomach and/or small intestine, providing for the induction of satiety and/or satiation.

Additional ingredients can be hydrophilic in nature, such as starch, protein, maltodextrin, and inulin. Other additional ingredients can be insoluble in water (e.g., cocoa solids, corn fiber) and/or fat soluble (vegetable oil), or can be flavor modifiers, such as, sucralose. For example, an extruded food product can include from about 5 to about 80% of a cereal ingredient, such as about 40% to about 68% of a cereal ingredient. A cereal ingredient can be rice, corn, wheat, sorghum, oat, or barley grains, flours, or meals. Thus, an extruded food product can include about 40% to about 50%, about 50% to about 58%, about 52% to about 57%, or about 52%, about 53%, about 54%, about 55%, about 56%, or about 56.5% of a cereal ingredient. In one embodiment, about 56.5% of rice flour is included.

An ingestible composition can also include a protein source. A protein source can be included in the composition or in an extruded food product. For example, an extruded food product can include a protein source at about 2% to about 20% by weight, such as about 3% to about 8%, about 3% to about 5%, about 4% to about 7%, about 4% to about 6%, about 5% to about 7%, about 5% to about 15%, about 10% to about 18%, about 15% to about 20%, or about 8% to about 18% by weight. A protein can be any known to those having ordinary skill in the art, e.g., rice, milk, egg, wheat, whey, soy, gluten, or soy flour. In some cases, a protein source can be a concentrate or isolate form.

Soluble Source

The compositions and associated methods of this invention include a source of at least one soluble cation source in an amount sufficient to cause an increase in viscosity of the soluble anionic fiber. A source of at least one soluble cation may be incorporated into an ingestible composition provided herein, or can consumed as a separate food article either before, after, or simultaneously with an ingestible composition.

Any soluble cation maybe used in the present invention, e.g., divalent, trivalent and the like. Cations useful in this invention include, calcium, magnesium, aluminum, manganese, iron, nickel, copper, zinc, strontium, barium, bismuth, chromium, vanadium, lanthanum, their salts and mixtures thereof. Soluble cation sources may be organic acid salts that include formate, fumarate, acetate, propionate, butyrate, caprylate, valerate, lactate, citrate, malate and gluconate. Also included are highly soluble inorganic salts such as chlorides or other halide salts.

In certain compositions, at least one soluble cations source is used with certain soluble anionic fibers, depending on the composition and gel strength desired. For example, for ingestible alginate compositions, calcium may be used to promote gellation. For gellan compositions, one or more of calcium and magnesium may be used.

The at least one soluble cation source can be unable to, or be limited in its ability to, react with the at least one soluble anionic fiber in the ingestible composition until during or after ingestion. For example, physical separation of the at least one soluble cation source from the at least one soluble anionic fiber, e.g., as a separate food article or in a separate matrix of the ingestible composition from the at least one soluble anionic fiber, can be used to limit at least one soluble cation sources ability to react. In other cases, the at least one soluble cation source is limited in its ability to react with the at least one soluble anionic fiber by protecting the source of at least one soluble cation source until during or after ingestion. Thus, the at least one soluble cation source, such as, a protected soluble cation source can be included in the ingestible composition or can be included as a separate food article composition, e.g., for separate ingestion either before, during, or after ingestion of an ingestible composition.

Typically, a separate food article containing the at least one soluble cation source would be consumed in an about four hour time window flanking the ingestion of an ingestible composition containing the at least one soluble anionic fiber. In certain cases, the window may be about three hours, or about two hours, or about one hour. In other cases, the separate food article may be consumed immediately before or immediately after ingestion of an ingestible composition, e.g., within about fifteen minutes, such as within about 10 mins., about 5 mins., or about 2 mins. In other cases, a separate food article containing at least one soluble cation source can be ingested simultaneously with an ingestible composition containing the at least one soluble anionic fiber, e.g., a snack chip composition where some chips include at least one soluble cation and some chips include the at least one soluble anionic fiber.

In one embodiment, at least one soluble cation source can be included in an ingestible composition in a different food matrix from a matrix containing an soluble anionic fiber. For example, a source of at least one soluble cation source, such as a soluble calcium salt, can be included in a separate matrix of a solid ingestible composition from the matrix containing the at least one soluble anionic fibers. Thus, means for physical separation of an soluble anionic fiber (e.g., within a snack bar or other extruded food product) from a source of at least one soluble cation source are also contemplated, such as by including the source of at least one soluble cation source in a matrix such as a frosting, water and fat based icing, coating, decorative topping, drizzle, chip, chunk, swirl, filling, or interior layer. In one embodiment, the at least one soluble cation source, such as, a protected soluble cation source, can be included in a snack bar matrix that also contains an extruded crispy matrix that contains the soluble anionic fiber. In such a case, the at least one soluble cation source is in a separate matrix than the extruded crispy matrix containing the soluble anionic fiber. In another embodiment, the at least one soluble cation source can be included in a gel layer or phase, e.g., a jelly or jam.

One soluble cation source is multivalent cation salts. Typically, a multivalent cation salt can be selected from the following salts: citrate, tartrate, malate, formate, lactate, gluconate, phosphate, carbonate, sulfate, chloride, acetate, propionate, butyrate, caprylate, valerate, fumarate, adipate, and succinate. In certain cases, a multivalent cation salt is a calcium salt. A calcium salt can have a solubility of >1% w/vol in water at pH 7 at 20° C. A calcium salt can be, without limitation, calcium citrate, calcium tartrate, calcium malate, calcium lactate, calcium gluconate, calcium citrate malate, dicalcium phosphate dihydrate, anhydrous calcium diphosphate, dicalcium phosphate anhydrous, calcium carbonate, calcium sulfate dihydrate, calcium sulfate anhydrous, calcium chloride, calcium acetate monohydrate, monocalcium phosphate monohydrate, and monocalcium phosphate anhydrous.

The source of at least one soluble cation source can be a protected source.

A number of methods can be used to protect at least one soluble cation source. For example, microparticles or nanoparticles having double or multiple emulsions, such as water/oil/water (“w/o/w”) or oil/water/oil (“o/w/o”) emulsions, of at least one soluble cation source and an soluble anionic fiber can be used. In one embodiment, a calcium alginate microparticle or nanoparticle is used. For example, a calcium chloride solution can be emulsified in oil, which emulsion can then be dispersed in a continuous water phase containing the anionic alginate soluble fiber. When the emulsion breaks in the stomach, the calcium can react with the alginate to form a gel.

A microparticle can have a size from about 1 to about 15 μM (e.g., about 5 to about 10 μM, or about 3 to about 8 μM). A nanoparticle can have a size of about 11 to about 85 nm (e.g., about 15 to about 50 nm, about 30 to about 80 nm, or about 50 to about 75 nm). The preparation of multiple or double emulsions, including the choice of surfactants and lipids, is known to those having ordinary skill in the art.

In another embodiment, nanoparticles of calcium alginate are formed by preparing nanodroplet w/o microemulsions of CaCl₂ in a solvent and nanodroplet w/o microemulsions of alginate in the same solvent. When the two microemulsions are mixed, nanoparticles of calcium alginate are formed. The particles can be collected and dispersed, e.g., in a fluid ingestible composition. As the particle size is small (<100 nm), the particles stay dispersed (e.g., by Brownian motion), or can be stabilized with a food grade surfactant. Upon ingestion, the particles aggregate and gel.

In other embodiments, a liposome containing a source of at least one multivalent cation can be included in an ingestible composition. For example, a calcium-containing liposome can be used. The preparation of liposomes containing multivalent cations is well known to those having ordinary skill in the art; see ACS Symposium Series, 1998 709:203-211; Chem. Mater. 1998 (109-116). Cochelates can also be used, e.g., as described in U.S. Pat. No. 6,592,894 and U.S. Pat. No. 6,153,217. The creation of cochelates using multivalent cations such as calcium can protect the multivalent cations from reacting with the soluble anionic fiber within the aqueous phase of an ingestible composition, e.g., by wrapping the multivalent cations in a hydrophobic lipid layer, thus delaying reaction with the fiber until digestion of the protective lipids in the stomach and/or small intestine via the action of lipases.

In certain cases, a soluble cation source containing carbohydrate glass can be used, such as a calcium containing carbohydrate glass. A carbohydrate glass can be formed from any carbohydrate such as, without limitation, sucrose, trehalose, inulin, maltodextrin, corn syrup, fructose, dextrose, and other mono-, di-, or oligo-saccharides using methods known to those having ordinary skill in the art; see, e.g., WO 02/05667. A carbohydrate glass can be used, e.g., in a coating or within a food matrix.

Ingestible Compositions

Compositions of the present invention can be in any form, fluid or solid. Fluids can be beverages, including shake, liquado, and smoothie. Fluids can be from low to high viscosity.

Solid forms can extruded or not. Solid forms may include bread, cracker, bar, mini-bars, cookie, confectioneries, e.g., nougats, toffees, fudge, caramels, hard candy enrobed soft core, muffins, cookies, brownies, cereals, chips, snack foods, bagels, chews, crispies, and nougats, pudding, jelly, and jam. Solids can have densities from low to high.

Fluid

Fluid ingestible compositions can be useful for, among other things, aiding in weight loss programs, e.g., as meal replacement beverages or diet drinks. Fluid ingestible compositions can provide from about 0.5 g to about 10 g of soluble anionic fiber per serving, or any value therebetween. For example, in certain cases, about 1 g, 2 g, 3 g, 4 g, 5 g, 6 g, 7 g, 8 g, or 9 g of at least one soluble anionic fiber are provided per serving.

A fluid ingestible composition may include an alginate soluble anionic fiber and/or a pectin soluble anionic fiber. In certain cases, an alginate soluble anionic fiber and a pectin soluble anionic fiber are used. A fiber blend as described herein can be used to provide the alginate soluble anionic fiber and/or the pectin soluble anionic fiber. An alginate and pectin can be any type and in any form, as described previously. For example, an alginate can be a high, medium, or low molecular weight range alginate, and a pectin can be a high-methoxy pectin. Also as indicated previously, two or more alginate forms can be used, such as a high molecular weight and a low molecular weight alginate, or two high molecular weight alginates, or two low molecular weight alginates, or a low and a medium molecular weight alginate, etc. For example, Manugel GHB alginate and/or Manugel LBA alginate can be used. In other cases, Manugel DPB can be used. Genu Pectin, USPL200 (a high-methoxy pectin) can be used as a pectin. In certain cases, potassium salt forms of an soluble anionic fiber can be used, e.g., to reduce the sodium content of an ingestible composition.

A fluid ingestible composition includes alginate and/or pectin in a total amount of about 1.0% to about 5% by weight, or any value therebetween, e.g., about 1.25% to about 1.9%; about 1.4% to about 1.8%; about 1.0% to about 2.2%, about 2.0% to about 4.0%, about 3.0%, about 4.0%, about 2.0%, about 1.5%, or about 1.5% to about 1.7%. Such percentages of total alginate and pectin can yield about 2 g to about 8 g of fiber per 8 oz. serving, e.g., about 3 g, about 4 g, about 5 g, about 6 g, or about 7 g fiber per 8 oz. serving. In other cases, about 4 g to about 8 g of fiber (e.g., about 5 g, about 6 g, or about 7 g) per 12 oz. serving can be targeted. In some embodiments, about 1.7% fiber by weight of a fluid ingestible composition is targeted.

In some cases, a fluid ingestible composition includes only alginate as a soluble anionic fiber. In other cases, alginate and pectin are used. A ratio of alginate to pectin (e.g., total alginate to total pectin) in a fluid ingestible composition can range from about 8:1 to about 1:8, and any ratio therebetween (e.g., alginate:pectin can be in a ratio of about 1:1, about 1.2:1, about 1.3:1, about 1.4:1, about 1.5:1, about 1.6:1, about 1.62:1, about 1.7:1, about 1.8:1, about 1.9:1, about 2:1, about 3:1, about 4:1, about 5:1, about 5.3:1, about 5.6:1, about 5.7:1, about 5.8:1, about 5.9:1, about 6:1, about 6.1:1, about 6.5:1, about 7:1, about 7.5:1, about 7.8:1, about 2:3, about 1:4, or about 0.88:1). In cases where alginate and pectin are in a ratio of about 0.5:1 to about 2:1, it is believed that pectin and alginate electrostatically associate with one another to gel in the absence of multivalent cations; thus, while not being bound by theory, it may be useful to delay the introduction of cations until after such gel formation. In other cases, where the ratio of alginate to pectin is in the range from about 3:1 to about 8:1, it may be useful to include a soluble cation source such as a calcium source (e.g., to crosslink the excess alginate) to aid gel formation in the stomach. In these cases, the inventors believe, while not being bound by any theory, that the lower amount of pectin protects the alginate from precipitating as algniate at the low pHs of the stomach environment, while the soluble cation source cross-links and stabilizes the gels formed.

A fluid ingestible composition can have a pH from about 3.9 to about 4.5, e.g., about 4.0 to about 4.3 or about 4.1 to about 4.2. At these pHs, it is believed that the fluid ingestible compositions are above the pKas of the alginate and pectin acidic subunits, minimizing precipitation, separation, and viscosity of the solutions. In some cases, malic, phosphoric, and citric acids can be used to acidify the compositions. In some cases, a fluid ingestible composition can have a pH of from about 5 to about 7.5. Such fluid ingestible compositions can use pH buffers known to those having ordinary skill in the art.

Sweeteners for use in a fluid ingestible composition can vary according to the use of the composition. For diet beverages, low glycemic sweeteners may be preferred, including trehalose, isomaltulose, aspartame, saccharine, and sucralose. Sucralose can be used alone in certain formulations. The choice of sweetener will impact the overall caloric content of a fluid ingestible composition. In certain cases, a fluid ingestible compositions can be targeted to have 40 calories/12 oz serving.

A fluid ingestible composition can demonstrate gel strengths of about 20 to about 250 grams force (e.g., about 60 to about 240, about 150 to about 240, about 20 to 30, about 20 to about 55, about 50 to 200; about 100 to 200; and about 175 to 240), as measured in a static gel strength assay. Gel strengths can be measured in the presence and absence of a soluble cation source, such as a calcium source.

A fluid ingestible composition can exhibit a viscosity in the range of from about 15 to about 100 cPs, or any value therebetween, at a shear rate of about 10⁻⁵, e.g., about 17 to about 24; about 20 to about 25; about 50 to 100, about 25 to 75, about 20 to 80, or about 15 to about 20 cPs. Viscosity can be measured by those skilled in the art, e.g., by measuring flow curves of solutions with increasing shear rate using a double gap concentric cyclinder fixture (e.g., with a Parr Physica Rheometer).

A fluid ingestible composition can include a cation sequestrant, e.g., to prevent premature gellation of the soluble anionic fibers. A multivalent cation sequestrant can be selected from EDTA and its salts, EGTA and its salts, sodium citrate, sodium hexametaphosphate, sodium acid pyrophosphate, trisodium phosphate anhydrous, tetrasodium pyrophosphate, sodium tripolyphosphate, disodium phosphate, sodium carbonate, and potassium citrate. A cation sequestrant can be from about 0.001% to about 0.3% by weight of the ingestible composition. Thus, for example, EDTA can be used at about 0.0015% to about 0.002% by weight of the ingestible composition and sodium citrate at about 0.230% to about 0.260% (e.g., 0.250%) by weight of the ingestible composition.

A fluid ingestible composition can include a juice or juice concentrate and optional flavorants and/or colorants. Juices for use include fruit juices such as apple, grape, raspberry, blueberry, cherry, pear, orange, melon, plum, lemon, lime, kiwi, passionfruit, blackberry, peach, mango, guava, pineapple, grapefruit, and others known to those skilled in the art. Vegetable juices for use include tomato, spinach, wheatgrass, cucumber, carrot, peppers, beet, and others known to those skilled in the art.

The brix of the juice or juice concentrate can be in the range of from about 15 to about 85 degrees, such as about 25 to about 50 degrees, about 40 to about 50 degrees, about 15 to about 30 degrees, about 65 to about 75 degrees, or about 70 degrees. A fluid ingestible composition can have a final brix of about 2 to about 25 degrees, e.g., about 5, about 10, about 12, about 15, about 20, about 2.5, about 3, about 3.5, about 3.8, about 4, or about 4.5.

Flavorants can be included depending on the desired final flavor, and include flavors such as kiwi, passionfruit, pineapple, coconut, lime, creamy shake, peach, pink grapefruit, peach grapefruit, pina colada, grape, banana, chocolate, vanilla, cinnamon, apple, orange, lemon, cherry, berry, blueberry, blackberry, apple, strawberry, raspberry, melon(s), coffee, and others, available from David Michael, Givaudan, Duckworth, and other sources.

Colorants can also be included depending on the final color to be achieved, in amounts quantum satis that can be determined by one having ordinary skill in the art.

Rapid gelling occurs when soluble anionic fibers, such as algniate or pectin, are mixed with soluble calcium sources, particularly the calcium salts of organic acids such as lactic or citric acid. For beverage products, this reactivity prevents the administration of soluble anionic fiber and a highly soluble calcium source in the same beverage. In the present invention, this problem is overcome by administering the soluble anionic fiber and the soluble calcium source in different product components.

Solids

At least one soluble anionic fiber and/or at least one soluble cation source can be present in a solid ingestible composition in any form or in any mixtures of forms. A form can be a processed, unprocessed, or both. Processed forms include extruded forms, spray-dried forms, roll-dried forms, or dry-blended forms. For example, a snack bar can include at least soluble anionic anionic fiber present as an extruded food product (e.g., a crispy), at least one soluble cation source in an unextruded form (e.g., as part of the bar), or both.

An extruded food product can be cold- or hot-extruded and can assume any type of extruded form, including without limitation, a bar, cookie, bagel, crispy, puff, curl, crunch, ball, flake, square, nugget, and snack chip. In some cases, an extruded food product is in bar form, such as a snack bar or granola bar. In some cases, an extruded food product is in cookie form. In other cases, an extruded food product is in a form such as a crispy, puff, flake, curl, ball, crunch, nugget, chip, square, chip, or nugget. Such extruded food products can be eaten as is, e.g., cookies, bars, chips, and crispies (as a breakfast cereal) or can be incorporated into a solid ingestible composition, e.g., crispies incorporated into snack bars.

A solid form may also be a lollipop or a lolly that is made of hardened, flavored sugar mounted on a stick and intended for sucking or licking. One form of lollipop has a soft-chewy filling in the center of the hardened sugar. The soft filling may be a gum, fudge, toffee, caramel, jam, jelly or any other soft-chewy filling known in the art. The at least one multivalent cation may be in the soft-chewy center or the harnend sugar. Likewise, at least fiber may be in the soft-chewy center or the harnend sugar. A hard candy filled with a soft-chewy center is another embodiment of the present invention. This embodiment is similar to the lollipop, except it is not mounted on a stick. The soft-chewy filling may be in the center or swirled or layered with the hard sugar confection.

A cookie or mini-bar can include at least one soluble anionic fiber in an unformed form or in a formed (e.g., extruded) form. A snack chip can include at least one soluble anionic fiber in extruded form or in spray-dried form, or both, e.g., an extruded soluble anionic fiber-containing chip having at least one soluble anionic fiber spray-dried on the chip.

A solid ingestible composition can include optional additions such as, frostings, icings, coatings, toppings, drizzles, chips, chunks, swirls, or layers. Such optional additions can include at least one soluble cation source, at least one soluble anionic fiber, or both.

Solid ingestible compositions can provide any amount from about 0.5 g to about 10 g total soluble anionic fiber per serving, e.g., about 0.5 g to about 5 g, about 1 g to about 6 g, about 3 g to about 7 g, about 5 g to about 9 g, or about 4 g to about 6 g. For example, in some cases, about 1 g, about 2 g, about 3 g, about 4 g, about 5 g, about 6 g, about 7 g, about 8 g, or about 9 g of soluble anionic fiber per serving can be provided.

A solid ingestible composition can include at least one soluble anionic fiber at a total weight percent of the ingestible composition of from about 4% to about 50% or any value therebetween. For example, a solid ingestible composition can include at least one soluble anionic fiber of from about 4% to about 10% by weight; or about 5% to about 15% by weight; or about 10% to about 20% by weight; or about 20% to about 30% by weight; or about 30% to about 40% by weight; or about 40% to about 50% by weight.

An extruded food product can be from about 0% to 100% by weight of an ingestible composition, or any value therebetween (about 1% to about 5%; about 5% to about 10%; about 10% to about 20%; about 20% to about 40%; about 30% to about 42%; about 35% to about 41%; about 37% to about 42%; about 42% to about 46%; about 30% to about 35%; about 40% to about 50%; about 50% to about 60%; about 60% to about 70%; about 70% to about 80%; about 80% to about 90%; about 90% to about 95%; about 98%; or about 99%). For example, an extruded bar, cookie, or chip can be about 80% to about 100% by weight of an ingestible composition or any value therebetween.

Alternatively, an ingestible composition can include about 30% to about 55% by weight of an extruded food product or any value therebetween, e.g., about 32%, about 33%, about 34%, about 35%, about 36%, about 37%, 3 about 8%, about 39%, about 40%, about 42%, about 45%, about 48%, about 50%, about 52%, or about 54% by weight of an extruded food product. For example, a snack bar composition can include extruded crispies in an amount of from about 32% to about 46% by weight of the snack bar.

Crispies

An extruded food product, e.g., for inclusion in an ingestible composition, can be a crispy. For example, crispies that include one or more alginates and/or pectins in a total amount of about 30% to about 35% by weight can be included in a snack bar in an amount of about 32% to about 45% by weight of the snack bar. Crispies can be prepared using a fiber blend as described herein. Crispies can also include, among other things, about 52% to about 58% by weight of one or more of a rice flour, corn meal, and/or corn cone; and about 2% to about 10% of a protein isolate. Crispies can be prepared using methods known to those having ordinary skill in the art, including cold and hot extrusion techniques.

An ingestible composition or formed food product can include one or more of the following: cocoa, including flavonols, and oils derived from animal or vegetable sources, e.g., soybean oil, canola oil, corn oil, safflower oil, sunflower oil, etc. For example, a formed food product can include cocoa or oils in an amount of about 3% to about 10% (e.g., about 3% to about 6%, about 4% to about 6%, about 5%, about 6%, about 7%, or about 4% to about 8%) by weight of the food product.

One embodiment of the present invention is a stable two phase product having at least one soluble anionic fiber and at least one soluble cation source in the same product, but formulated so that the soluble anionic fiber and cation source do not react during processing or prior to ingestion, but react following ingestion as a standard cation-anion reaction. One product design includes a jam phase center, e.g., fluid phase, and a crisp baked phase, e.g., solid phase, outside the jam phase. One embodiment places the soluble anionic fiber in the jam phase and places the soluble cation source in the baked phase.

Another embodiment of the present invention addresses this issue, adding of the soluble anionic fiber to the baked dough phase and the multivalent cation to the jam phase, which provides a cookie that reduces the water activity of the fiber-containing phase which restricted fiber so that it was prevented from reacting with the multivalent cation. The placement of the multivalent cation into a postbake, medium water activity filler, e.g., the fluid phase, allowed the cation to be formulated in the product with an acceptable organoleptic profile and an inability to react with fiber even if minor migration occurs.

The water activities of both components can be further adjusted to deliver a product with not only restrictive reaction in place but acceptable eating qualities and the right characteristics needed to for ease of manufacturing.

Types of salts tested include calcium fumarate, tricalcium phosphate, dicalcium phosphate dihydrate and calcium carbonate. The gram weight tested will vary depending on the salt type due to its characteristic calcium load. The piece weight of the product under discussion has been about 13 to about 20 g, with each piece delivering from about 50 to about 75 kcal.

BENEFAT® is a family of triglyceride blends made from the short and long chain fatty acids commonly present in the diet. It is the uniqueness of these fatty acids that contribute to the range's reduced calorie claim. BENEFAT® products are designed to replace conventional fats and oils in dairy, confectionery and bakery products, giving full functionality with significantly reduced energy and fat content. BENEFAT® is the Danisco trade name for SALATRIM, the abbreviation for short and long-chain triglyceride molecules. The short-chain acids (C₂-C₄) may be acetic, propionic, butyric or a combination of all three, while the long-chain fatty acid (C₁₆-C₂₂) is predominantly stearic and derived from fully hardened vegetable oil. Unlike other saturated fatty acids, stearic acid has a neutral effect on blood cholesterol. BENEFAT® is also free of trans fatty acids and highly resistant to oxidation. Compared to the 9 calories per gram of traditional fat, BENEFAT® contains just 5 calories per gram (US regulation) or 6 calories per gram (EU regulation), at the same time giving foods a similar creamy taste, texture, and mouthfeel as full-fat products. Metabolisation upon consumption occurs in much the same way as with other food components.

A preferred product features include about 500 to about 1500 mg of alginate are present, the cation is calcium, wherein about 50 to about 500 mg of elemental calcium are delivered. The product has low calories between about 50 to about 100 calories and is a cookie with a jam filling.

The soluble anionic fiber can be provided in one beverage component, and a soluble cation source can be provided in a second beverage component. The first component and the second component are provided separately to the user in a bottle or cup, and the user consumes the two components concurrently or sequentially.

Alternately, the soluble anionic fiber may be delivered in a beverage component and a soluble cation source may be provided separately in a solid edible component. The fluid fiber component and the solid soluble cation source component are consumed concurrently or sequentially.

The soluble anionic fiber component may also be provided in a solid edible component, and the soluble cation source may be provided separately in a fluid component. The fluid soluble cation source component and the solid fiber component are consumed concurrently or sequentially.

The soluble anionic fiber component and the soluble cation source can both be provided in solid edible components. The components can be provided in the form of separate items for consumption, or both components can be combined in a single solid form for consumption. This single solid form may contain the soluble anionic fiber in one phase, such as, a layer or filling, and the soluble cation source can be provided in a separate phase, such as a layer or filling. Alternatively, the fiber and soluble cation source may be intimately mixed in the same solid form.

The ingestible composition of the present invention can be provided in any package, such as enclosed in a wrapper or included in a container. An ingestible composition can be included in an article of manufacture. An article of manufacture that includes an ingestible composition described herein can include auxiliary items such as straws, napkins, labels, packaging, utensils, etc.

An article of manufacture can include a source of at least one soluble cation source. For example, the soluble cation source can be provided as a fluid, e.g., as a beverage to be consumed before, during, or after ingestion of the ingestible composition. In other cases, at least one soluble cation source can be provided in a solid or gel form. For example, a source of at least one multivalent cation can be provided in, e.g., a jelly, jam, dip, swirl, filling, or pudding, to be eaten before, during, or after ingestion of the ingestible composition. Thus, in some embodiments, an article of manufacture that includes a cookie or bar solid ingestible composition can also include a dip comprising a source of at least one soluble cation source, e.g., into which to dip the cookie or bar solid ingestible composition.

Also provided are articles of manufacture that include a fluid ingestible composition. For example, a fluid ingestible composition can be provided in a container. Supplementary items such as straws, packaging, labels, etc. can also be included. Alternatively, the soluble anionic fiber may be included in a beverage and the soluble cation source may be provided inside, outside or both of a straw or stirring stick. In some cases, at least one soluble cation source, as described below, can be included in an article of manufacture. For example, an article of manufacture can include a fluid ingestible composition in one container, and a soluble cation source in another container. Two or more containers may be attached to one another.

Methods of Reducing Caloric Consumption

An soluble anionic fiber (such as algniate and pectin) is orally administered concurrently with a soluble cation source, such as, a water-soluble calcium salt, to reduce caloric intake. Continued use of these compositions by individuals in need of weight loss will result in a cumulative decrease in caloric consumption, which will result in weight loss or diminished weight gain. Although not wishing to be bound by theory, the inventors hypothesize that the cation source cross link the carboxylate groups on the fiber molecules, resulting in the formation of highly viscous or gelled materials. This gelling effect increases the viscosity of the gastric and intestinal contents, slowing gastric emptying, and also slowing the rate of macronutrient, e.g., glucose, amino acids, fatty acids, and the like, absorption. These physiological effects prolong the period of nutrient absorption after a meal, and therefore prolong the period during which the individual experiences an absence of hunger. The increased viscosity of the gastrointestinal contents, as a result of the slowed nutrient absorption, also causes a distal shift in the location of nutrient absorption. This distal shift in absorption may trigger the so-called “ileal brake”, and the distal shift may also cause in increase in the production of satiety hormones such as GLP-1 and PYY.

Provided herein are methods employing the ingestible compositions described herein. For example, a method of inducing satiety and/or satiation in an animal is provided. The method can include orally administering an ingestible composition to an animal. An animal can be any animal, including a human, monkey, mouse, rat, snake, cat, dog, pig, cow, sheep, bird or horse. Oral administration can include providing the ingestible combination either alone or in combination with other meal items. Oral administration can include co-administering, either before, after, or during administration of the ingestible composition, a cation source, such as calcium or a sequestered source of calcium, as described herein. At least one soluble cation source can be administered within about a four hour time window flanking the administration of the ingestible composition. For example, a soluble calcium source such as a solution of calcium lactate, can be administered to an animal immediately after the animal has ingested a fluid ingestible composition as provided herein. Satiety and/or satiation can be evaluated using consumer surveys (e.g., for humans) that can demonstrate a statistically significant measure of increased satiation and/or satiety. Alternatively, data from paired animal sets showing a statistically significant reduction in =total caloric intake or food intake in the animals administered the ingestible compositions can be used as a measure of facilitating satiety and/or satiation.

As indicated previously, the ingestible compositions provide herein can hydrate and gel in the stomach and/or small intestine, leading to increased viscosity in the stomach and/or small intestine after ingestion. Accordingly, provided herein are methods for increasing the viscosity of stomach and/or small intestine contents, which include administering an ingestible composition to an animal. An animal can be any animal, as described above, and administration can be as described previously. Viscosity of stomach contents can be measured by any method known to those having ordinary skill in the art, including endoscopic techniques, imaging techniques (e.g., MRI), or in vivo or ex vivo viscosity measurements in e.g., control and treated animals.

Also provided are methods for promoting weight loss by administering an ingestible composition as provided herein to an animal. Administration can be as described previously. The amount and duration of such administration will depend on the individual's weight loss needs and health status, and can be evaluated by those having ordinary skill in the art. The animal's weight loss can be measured over time to determine if weight loss is occurring. Weight loss can be compared to a control animal not administered the ingestible composition.

The following examples are representative of the invention, and are not intended to be limiting to the scope of the invention.

### EXAMPLES

### Example 1

A cookie having a solid phase, e.g., a baked dough phase, containing a soluble anionic fiber blend and a fluid phase, e.g., jam phase containing a soluble calcium source deposited in the baked dough phase was produced.

The baked dough phase was prepared by adding BENEFAT® and lecithin to a premix of flour, cellulose, egg white, salt, leavening and flavors in a Hobart mixer and creaming by mixing at low speed for about 1 minute followed by high speed for about 2 minutes. The liquids were added to creamed mixture and blended at medium speed for about 2 minutes.

The fiber blend used contained about 46% sodium alginate LBA (ISP, San Diego, Calif.), about 39.6% sodium alginate GHB (ISP), and about 14.4% pectin (USP-L200, Kelco, San Diego, Calif.).

The fiber blend and glycerin were added to a separate bowl and combined. This combined fiber/glycerin material was added to the other ingredients in the Hobart mixer and was mixed on medium speed for about 1 minute. The resulting dough was then sheeted to desired thickness on a Rhondo sheeter and a dough pad measuring about 3 inched by about 6 inches was created.

The jam phase was prepared by adding a premixed BENEFAT®/calcium source mixture to the jam base and mixed until uniformly mixed. A predetermined amount of the jam was then added onto the top surface of the cookie dough pad. The dough pad edges were wetted and sealed. Bars were baked at 325° F. for about 9 minutes, cut, cooled and the resulting cookies were individually packaged. The total caloric value of each cookie was about 50 kcal.

| | % Dough | % Total |
|---|---|---|
| Ingredient | Phase | Formulation |
| flour all purpose | 29.140 | 12.165 |
| cellulose, solka floc - | 6.980 | 2.914 |
| International Fiber Corp. | | |
| Powder egg white | 0.580 | 0.242 |
| salt (NaCl) | 0.200 | 0.083 |
| Sodium Bicarbonate Grade #1 | 0.510 | 0.213 |
| cookie dough flavor | 0.170 | 0.071 |
| BENEFAT | 2.060 | 0.860 |
| Lecithin | 0.640 | 0.267 |
| polydextrose litesse 70% syrup, | 15.870 | 6.625 |
| Ultra | | |
| Water | 11.830 | 4.939 |
| liquid vanilla flavor | 0.280 | 0.117 |
| sucralose, 25% liquid. | 0.090 | 0.038 |
| potassium sorbate | 0.250 | 0.104 |
| Alginate fiber blend | 17.400 | 7.264 |
| glycerine, Optim 99.7% USP | 14.000 | 5.845 |
| | 100.000 | 41.70 |

Jam Phase:

| | % Jam | % Total |
|---|---|---|
| Ingredient | Phase | Formulation |
| BENEFAT | 21.100 | 12.291 |
| Calcium fumarate trihydrate | 11.000 | 6.408 |
| reduced calorie strawberry Filling | 67.900 | 39.553 |
| (Smuckers) | | |
| | 100.000 | 58.25 |

Control

Dough Phase:

| | % Dough | % Total |
|---|---|---|
| Ingredient | Phase | Formulation |
| Flour all purpose | 29.140 | 12.530 |
| Cellulose, solka floc - | 6.980 | 3.001 |
| International Fiber Corp. | | |
| Powder egg white | 0.580 | 0.249 |
| Salt (NaCl) | 0.200 | 0.086 |
| Sodium Bicarbonate Grade #1 | 0.510 | 0.219 |
| Cookie Dough Flavor | 0.170 | 0.073 |
| BENEFAT | 19.450 | 8.364 |
| Lecithin | 0.640 | 0.275 |
| Polydextrose Litesse 70% syrup, | 15.870 | 6.824 |
| Ultra | | |
| Water | 11.830 | 5.087 |
| Liquid Vanilla flavor | 0.280 | 0.120 |
| sucralose, 25% liquid. | 0.090 | 0.039 |
| Potassium sorbate | 0.250 | 0.108 |
| Alginate fiber blend | 0.000 | 0.000 |
| Glycerine, Optim 99.7% USP | 14.000 | 6.020 |
| | 100.000 | 43.00 |

Jam Phase:

| | | % Total |
|---|---|---|
| Ingredient | % Jam Phase | Formulations |
| BENEFAT | 32.100 | 19.260 |
| Reduced Calorie | 67.900 | 40.740 |
| Strawberry Filling | | |
| (SMUCKERS) | | |
| Total | 100.000 | 60.00 |

Measurement of Intestinal Viscosity

Fully grown female Yucatan minipigs (Charles River Laboratories, Wilmington, Mass.), weighing about 90 kg, were fitted with indwelling silicone rubber sample ports (Omni Technologies, Inc., Greendale, Ind.) implanted in a surgically created dermal fistula at the ileocecal junction. The sample ports were sealed by a removable cap. These ports permit removal of samples of digesta as it passed from the ileum to the cecum. Additional details of this procedure are presented in B. Greenwood van-Meerveld et al., Comparison of Effects on Colonic Motility and Stool Characteristics Associated with Feeding Olestra and Wheat Bran to Ambulatory Mini-Pigs, Digestive Diseases and Sciences 44:1282-7 (1999), which is incorporated herein by reference.

Three Yucatan minipigs with the fistulas described above were housed in individual stainless steel pens in a windowless room maintained on a cycle of 12 hours of light and 12 hours of dark. They were conditioned to consume low fiber chow (Laboratory Mini-Pig Diet 5L80, PMI Nutritional International, Brentwood, Mo.). This chow contained about 5.3% fiber. The pigs were fed once each day, in the morning. Water was provided ad lib throughout the day.

Samples were taken from the ileal sample port immediately after feeding, and then at about 30 minute intervals for about 300 minutes. The volume of sample collected was about 50 to 130 ml. All samples were assayed for viscosity within 30 minutes after collection.

Samples of digesta were collected in sealed plastic containers. Viscosity of the digesta were measured with a Stevens QTS Texture Analyzer (Brookfield Engineering, Inc., Middleboro, Mass.). This instrument measured the relative viscosity of digesta by a back extrusion technique. The instrument included a stage plate, a 60 cm vertical tower, a mobile beam and a beam head that contained a load-cell. During back extrusion, the beam descended at a constant rate, and the force required to back extrude the sample was recorded over time. The sample containers were 5 cm deep spherical aluminum cups with an internal diameter of about 2.0 cm. The volume of the cup was about 20 ml. The spherical probe consisted of a 1.9 cm TEFLON ball mounted on a 2 mm threaded rod which was attached to the mobile beam. The diameters of the sample cup and probe allowed for a wide range of viscosity (liquid to solid digesta) to be measured without approaching the maximum capacity of the rheometer (25 kg/peak force). During each test, the beam thrusted the probe into the test sample at a constant rate (12 cm/second) for a 2 cm stroke, forcing the sample to back-extrude around the equatorial region of the probe. The peak force for back extrusion at a controlled stroke rate was proportional to the viscosity of the sample. At each time point, 2-6 samples from each pig were tested, and the mean peak force is calculated and recorded.

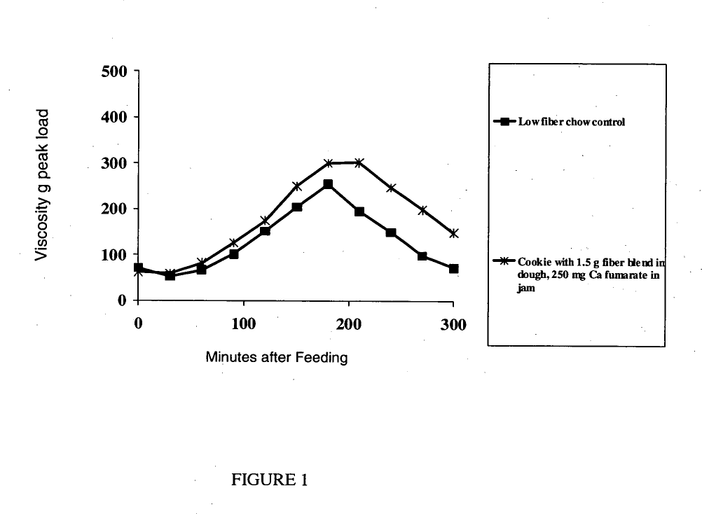

The test for effects of fiber containing cookies on viscosity was performed by providing each pig with its daily ration of low fiber chow (1400 g). Before feeding, one cookie was gently broken into four to six pieces and mixed into the chow. The animals had unlimited access to water during and after feeding. The effects of the cookie of this example containing fiber and calcium on intestinal viscosity is shown in FIG. 1. Each treatment was provided to each of three pigs on three separate days to yield nine replicates for each sample. Each point plotted in FIG. 1 is the mean of these nine determinations. The fiber and calcium containing cookie produced viscosities significantly greater than those produced by control chow (p<0.05, as measured by a two-tailed t-test) at the time points from 210 minutes through 300 minutes.

### Example 2—Bars

Nutritional bars with a nougat center were prepared by the following procedure. All liquid ingredients were placed in a mixer bowl with the paddle attachment. After one mixing for one minute, the dry ingredients were added except proteins and mixing was continued to mix on low speed. After 1 minute, proteins were added to the dough, and mixing was continued on low to medium speed for an additional 2 minutes. The dough was then formed into desired shapes and sizes either manually or through an extruder. Bars were coated with coatings of desired flavors and/or colors by submersion into melted (120° F.) compound coating, or into chocolate that has been melted (1200F) and tempered (900F). Coated bars were allowed to cool to harden the coating, and were then packaged.

Chocolate Peanut Butter
Serving size: 50 g

| # | Ingredient | Percentage |
|---|---|---|
| 1 | Chocolate Coating | 12.50 |
| 2 | HFCS | 20.00 |
| 3 | Glycerine | 12.50 |
| 4 | Water | 10.00 |
| 5 | Canola Oil | 5.00 |
| 6 | Inulin | 5.00 |
| 7 | Tricalcium Phosphate | 2.50 |
| 8 | Calcium Caseinate | 10.00 |
| 9 | Whey Protein Isolate | 12.50 |
| 10 | Psyllium | 10.00 |
| | Total | 100.00 |

Chocolate Peanut Butter
Formula # 5367-45-33
Serving size 55 g, 5 g alginate

| # | Ingredient | Percentage |
|---|---|---|
| 1 | Sugar-Free Choc Coating | 18.20 |
| 2 | High fructose corn syrup | 13.00 |
| 3 | Maltitol | 9.90 |
| 4 | Glycerine | 5.00 |
| 5 | Peanut Butter | 3.00 |
| 6 | Canola Oil | 3.00 |
| 7 | Peanut Butter Flavor | 3.00 |
| 8 | Vanilla | 0.50 |
| | Mix for 0.5 min | |
| 9 | Alginate | 9.10 |
| | Mix for 1 min | |
| 10 | Erythritol | 8.00 |
| 11 | Inulin | 3.00 |
| 12 | Peanut Flour | 5.00 |
| | Mix for 0.5 min | |
| 13 | Tricalcium Phosphate | 0.00 |
| 14 | Calcium Carbonate | 0.00 |
| 15 | Whey Protein Isolate | 8.30 |
| 16 | Peanuts | 11.00 |
| | Mix for 1 min | |
| | Total | 100.00 |

Chocolate Peanut Butter
Formula # 5367-45-32
Serving size 55 g, 5 g alginate

| # | Ingredient | Percentage |
|---|---|---|
| 1 | Sugar-Free Choc Coating | 18.20 |
| 2 | High fructose corn syrup | 12.00 |
| 3 | Maltitol | 9.90 |
| 4 | Glycerine | 3.00 |
| 5 | Peanut Butter | 3.00 |
| 6 | Peanut Butter Flavor | 3.00 |
| 7 | Vanilla | 0.50 |
| | Mix for 0.5 min | |
| 8 | Alginate | 9.10 |
| | Mix for 1 min | |
| 9 | Erythritol | 8.00 |
| 10 | Inulin | 5.00 |
| 11 | Peanut Flour | 5.00 |
| 12 | Hydrolyzed Whey Isolate | 4.00 |
| 13 | Sucrolose | 0.00 |
| | Mix for 0.5 min | |
| 14 | Tricalcium Phosphate | 0.00 |
| 15 | Calcium Carbonate | 0.00 |
| 16 | Whey Protein Isolate | 8.30 |
| 17 | Peanuts | 11.00 |
| | Mix for 1 min | |
| | Total | 100.00 |
| | Aw = 0.521 | |

Chocolate Peanut Butter
Formula # 5367-45-01
Serving size 45 g, contains 200 mg Ca, 5% alginate/pectin, 3 g inulin

| # | Ingredient | Percentage |
|---|---|---|
| 1 | Chocolate Coating | 15.00 |
| 2 | High fructose corn syrup | 9.50 |
| 3 | Water | 9.50 |
| 4 | Glycerine | 7.00 |
| 5 | Peanut Flour | 8.00 |
| 6 | Peanut Butter | 10.00 |
| 7 | Peanut Flavor | 1.15 |
| 8 | Inulin | 6.70 |
| 9 | Alginate | 2.50 |
| 10 | Pectin | 2.50 |
| 11 | Soy Protein Isolate | 8.00 |
| 12 | Calcium Caseinate | 7.00 |
| 13 | Whey Protein Isolate | 7.00 |
| 14 | Peanuts | 6.15 |
| 15 | Tricalcium Phosphate | 1.15 |
| | Total | 100.00 |

Chocolate Raspberry
Formula # 5367-44-03
Serving size 40 g, contains 200 mg Ca, 1 g alginate, 1 g pectin, 3 g inulin

| # | Ingredient | Percentage |
|---|---|---|
| 1 | Sugar-Free Choc Coating | 18.00 |
| 2 | Water | 12.60 |
| 3 | Glycerine | 8.00 |
| 4 | Canola Oil | 7.00 |
| 5 | Raspberry Flavor | 1.50 |
| 6 | Raspberry Flavor | 0.75 |
| 7 | White Chocolate Flavor | 0.35 |
| 8 | Vanilla Flavor | 1.00 |
| 9 | Sucrolose | 0.20 |
| 10 | Red Color | 0.10 |
| 11 | Inulin | 5.45 |
| 12 | Erythritol | 8.00 |
| 13 | Fructose | 4.00 |
| 14 | Malic Acid | 0.30 |
| 15 | Alginate | 2.15 |
| 16 | Pectin | 3.04 |
| 17 | Soy Protein Isolate | 5.50 |
| 18 | Calcium Caseinate | 5.50 |
| 19 | Whey Protein Isolate | 8.00 |
| 20 | Tricalcium Phosphate | 0.96 |
| 21 | Dried Raspberry | 3.00 |
| 22 | Soy Crisps, 80% protein | 4.60 |
| | Total | 100.00 |

Chocolate Peanut Butter
Formula # 5367-45-13
Serving size 55 g, no calcium, 5 g alginate

| # | Ingredient | Percentage |
|---|---|---|
| 1 | Sugar-Free Choc Coating | 20.00 |
| 2 | Corn Syrup | 14.50 |
| 3 | Glycerine | 12.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 9.40 |
| 17 | Soy Crisps | 0.00 |
| | Total | 100.00 |

Chocolate Peanut Butter
Formula # 5367-45-14
Serving size 55 g, no calcium, 5 g alginate
LBA alginate

| # | Ingredient | Percentage |
|---|---|---|
| 1 | Sugar-Free Choc Coating | 20.00 |
| 2 | Corn Syrup | 14.50 |
| 3 | Glycerine | 12.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 9.40 |
| 17 | Soy Crisps | 0.00 |
| | Total | 100.00 |

Aw = 0.383

Chocolate Peanut Butter
Formula # 5367-45-15
Serving size 55 g, no calcium, 5 g alginate
GHB alginate

| # | Ingredient | Percentage |
|---|---|---|
| 1 | Sugar-Free Choc Coating | 20.00 |
| 2 | Corn Syrup | 14.50 |
| 3 | Glycerine | 8.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 5.00 |
| 13 | Calcium Caseinate | 5.00 |
| 14 | Whey Protein Isolate | 5.00 |
| 15 | Peanuts | 10.40 |
| 16 | Soy Crisps | 0.00 |
| | Total | 100.00 |

Aw = 0.383

Chocolate Peanut Butter
Formula # 5367-45-16
Serving size 55 g, no calcium, 5 g alginate (LBA alginate)

| # | Ingredient | Percentage |
|---|---|---|
| 1 | Sugar-Free Choc Coating | 20.00 |
| 2 | Corn Syrup | 14.50 |
| 3 | Glycerine | 8.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 5.00 |
| 13 | Calcium Caseinate | 5.00 |
| 14 | Whey Protein Isolate | 5.00 |
| 15 | Peanuts | 10.40 |
| 16 | Soy Crisps | 0.00 |
| | Total | 100.00 |

Aw = 0.383

Chocolate Peanut Butter
Formula # 5367-45-17
Serving size 55 g, no calcium
Spray Dry Batch #1

| # | Ingredient | Percentage |
|---|---|---|
| 1 | Sugar-Free Choc Coating | 20.00 |
| 2 | Corn Syrup | 17.00 |
| 3 | Glycerine | 3.00 |
| 4 | Peanut Butter | 10.00 |
| 5 | Peanut Butter Flavor | 2.00 |
| 6 | Maltodextrin | 0.00 |
| 7 | Erythritol | 4.00 |
| 8 | Alginate | 21.81 |
| 9 | Pectin | 0.00 |
| 10 | Peanut Flour | 3.40 |
| 11 | Tricalcium Phosphate | 0.00 |
| 12 | Soy Protein Isolate | 3.50 |
| 13 | Calcium Caseinate | 3.50 |
| 14 | Whey Protein Isolate | 3.50 |
| 15 | Peanuts | 8.29 |
| 16 | Soy Crisps | 0.00 |
| | Total | 100.00 |

Aw 0.519

Chocolate Peanut Butter
Formula # 5367-45-18
Serving size 55 g, no calcium, 5 g alginate
LBA

| # | Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 10.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 3.00 |
| 13 | Calcium Caseinate | 3.00 |
| 14 | Whey Protein Isolate | 3.00 |
| 15 | Peanuts | 7.90 |
| 16 | Soy Crisps | 0.00 |
| | Total | 99.00 |

Aw = 0.340

Chocolate Peanut Butter
Formula # 5367-45-19
Serving size 55 g, no calcium
Spray Dry Batch # 2

| # | Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 10.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 3.00 |
| 13 | Calcium Caseinate | 3.00 |
| 14 | Whey Protein Isolate | 3.00 |
| 15 | Peanuts | 7.90 |
| 16 | Soy Crisps | 0.00 |
| | Total | 99.00 |

Aw = 0.340

Chocolate Peanut Butter
Formula # 5367-45-20
Serving size 55 g, no calcium
Spray Dry Batch # 3

| # | Ingredient | Percentage |
|---|---|---|
| 1 | Sugar-Free Choc Coating | 20.00 |
| 2 | Corn Syrup | 20.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 16.00 |
| 10 | Pectin | 0.00 |
| 11 | Peanut Flour | 2.30 |
| 12 | Tricalcium Phosphate | 0.00 |
| 13 | Soy Protein Isolate | 0.00 |
| 14 | Calcium Caseinate | 0.00 |
| 15 | Whey Protein Isolate | 0.00 |
| 16 | Peanuts | 8.00 |
| 17 | Soy Crisps | 0.00 |
| | Total | 95.00 |

Chocolate Peanut Butter
Formula # 5367-45-21
Serving size 55 g, no calcium
Spray Dry Batch # 4

| # | Ingredient | Percentage |
|---|---|---|
| 1 | Sugar-Free Choc Coating | 20.00 |
| 2 | Corn Syrup | 20.00 |
| 3 | Glycerine | 0.00 |
| | Maltitol | 16.10 |
| 4 | Peanut Butter | 10.00 |
| 5 | Peanut Butter Flavor | 2.00 |
| 6 | Maltodextrin | 0.00 |
| 7 | Erythritol | 0.00 |
| 8 | Alginate | 21.40 |
| 9 | Pectin | 0.00 |
| 10 | Peanut Flour | 2.30 |
| 11 | Tricalcium Phosphate | 0.00 |
| 12 | Soy Protein Isolate | 0.00 |
| 13 | Calcium Caseinate | 0.00 |
| 14 | Whey Protein Isolate | 0.00 |
| 15 | Peanuts | 3.20 |
| 16 | Soy Crisps | 0.00 |
| | Total | 95.00 |

Chocolate Peanut Butter
Formula # 5367-45-22
Serving size 55 g, no calcium
Spray Dry Batch # 5

| # | Ingredient | Percentage |
|---|---|---|
| 1 | Sugar-Free Choc Coating | 20.00 |
| 2 | Corn Syrup | 20.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 16.00 |
| 10 | Pectin | 0.00 |
| 11 | Peanut Flour | 2.30 |
| 12 | Tricalcium Phosphate | 0.00 |
| 13 | Soy Protein Isolate | 0.00 |
| 14 | Calcium Caseinate | 0.00 |
| 15 | Whey Protein Isolate | 0.00 |
| 16 | Peanuts | 8.00 |
| 17 | Soy Crisps | 0.00 |
| | Total | 95.00 |

Chocolate Peanut Butter
Formula # 5367-45-23
Serving size 55 g, no calcium
Spray Dry Batch # 6
17% coating

| # | Ingredient | Percentage |
|---|---|---|
| 1 | Sugar-Free Choc Coating | 20.00 |
| 2 | Corn Syrup | 20.00 |
| 3 | Glycerine | 0.00 |
| | Maltitol | 16.70 |
| 4 | Peanut Butter | 10.00 |
| 5 | Peanut Butter Flavor | 2.00 |
| 6 | Maltodextrin | 0.00 |
| 7 | Erythritol | 0.00 |
| 8 | Alginate | 16.00 |
| 9 | Pectin | 0.00 |
| 10 | Peanut Flour | 2.30 |
| 11 | Tricalcium Phosphate | 0.00 |
| 12 | Soy Protein Isolate | 0.00 |
| 13 | Calcium Caseinate | 0.00 |
| 14 | Whey Protein Isolate | 0.00 |
| 15 | Peanuts | 8.00 |
| 16 | Soy Crisps | 0.00 |
| | Total | 95.00 |

Chocolate Peanut Butter
Formula # 5367-45-24
Serving size 55 g, no calcium
Spray Dry Batch #1

| # | Ingredient | Percentage |
|---|---|---|
| 1 | Sugar-Free Choc Coating | 20.00 |
| 2 | Corn Syrup | 17.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 21.81 |
| 10 | Pectin | 0.00 |
| 11 | Peanut Flour | 3.40 |
| 12 | Tricalcium Phosphate | 0.00 |
| 13 | Soy Protein Isolate | 1.00 |
| 14 | Calcium Caseinate | 1.00 |
| 15 | Whey Protein Isolate | 1.00 |
| 16 | Peanuts | 6.09 |
| 17 | Soy Crisps | 0.00 |
| | Total | 100.00 |

Chocolate Peanut Butter
Formula # 5367-45-25
Serving size 55 g, 5 g alginate
LBA (alginate)

| # | Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

Chocolate Peanut Butter
Formula # 5367-45-26
Serving size 55 g, 5 g alginate

| # | Ingredient | Percentage |
|---|---|---|
| 1 | Sugar-Free Choc Coating | 20.00 |
| 2 | Corn Syrup | 16.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 102.00 |

Chocolate Peanut Butter, Jan. 26, 2004
Formula # 5367-45-27
Serving size 55 g, 5 g alginate

| # | Ingredient | Percentage |
|---|---|---|
| 1 | Sugar-Free Choc Coating | 20.00 |
| 2 | Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 5.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

Chocolate Peanut Butter
Formula # 5367-45-30B, LBA
Serving size 55 g, 5 g alginate

| # | Ingredient | Percentage |
|---|---|---|
| 1 | Sugar-Free Choc Coating | 18.20 |
| 2 | HFCS | 10.00 |
| 3 | Glycerine | 7.00 |
| 4 | Maltitol | 7.00 |
| 5 | Peanut Butter | 3.00 |
| 6 | Peanut Butter Flavor | 3.00 |
| 7 | Vanilla | 0.50 |
| 8 | Sucrolose | 0.01 |
| 9 | Inulin | 5.00 |
| 10 | Erythritol | 8.00 |
| 11 | Alginate | 9.10 |
| 12 | Peanut Flour | 5.00 |
| 13 | Hydrolyzed Whey Isolate | 7.00 |
| 14 | Tricalcium Phosphate | 0.00 |
| 15 | Calcium Carbonate | 0.00 |
| 16 | Whey Protein Isolate | 8.50 |
| 17 | Peanuts | 8.69 |
| | Total | 100.00 |
| | Aw = 0.402 | |

Chocolate Peanut Butter
Formula # 5367-45-28
Serving size 55 g, 5 g alginate

| # | Ingredient | Percentage |
|---|---|---|
| 1 | Sugar-Free Choc Coating | 20.00 |
| 2 | Corn Syrup | 20.00 |
| 3 | Glycerine | 6.00 |
| 4 | Maltitol | 5.00 |
| 5 | Peanut Butter | 8.00 |
| 6 | Peanut Butter Flavor | 2.00 |
| 7 | Maltodextrin | 0.00 |
| 8 | Inulin | 0.00 |
| 9 | Erythritol | 5.00 |
| 10 | Alginate | 0.00 |
| 11 | Pectin | 0.00 |
| 12 | Spray Dry Alginate | 14.26 |
| 13 | Peanut Flour | 3.40 |
| 14 | Tricalcium Phosphate | 0.00 |
| 15 | Calcium Carbonate | 0.00 |
| 16 | Soy Protein Isolate | 3.00 |
| 17 | Calcium Caseinate | 3.00 |
| 18 | Whey Protein Isolate | 3.00 |
| 19 | Peanuts | 7.34 |
| 20 | Soy Crisps | 0.00 |
| | Total | 100.00 |

Chocolate Peanut Butter
Formula # 5367-45-29
Serving size 55 g, 5 g alginate

| # | Ingredient | Percentage |
|---|---|---|
| 1 | Sugar-Free Choc Coating | 20.00 |
| 2 | Corn Syrup | 20.00 |
| 3 | Glycerine | 6.00 |
| 4 | Maltitol | 5.00 |
| 5 | Peanut Butter | 8.00 |
| 6 | Peanut Butter Flavor | 2.00 |
| 7 | Maltodextrin | 4.76 |
| 8 | Inulin | 0.00 |
| 9 | Erythritol | 5.00 |
| 10 | Alginate | 0.00 |
| 11 | Pectin | 0.00 |
| 12 | Spray Dry Alginate | 9.50 |
| 13 | Peanut Flour | 3.40 |
| 14 | Tricalcium Phosphate | 0.00 |
| 15 | Calcium Carbonate | 0.00 |
| 16 | Soy Protein Isolate | 3.00 |
| 17 | Calcium Caseinate | 3.00 |
| 18 | Whey Protein Isolate | 3.00 |
| 19 | Peanuts | 7.34 |
| 20 | Soy Crisps | 0.00 |
| | Total | 100.00 |

Chocolate Peanut Butter
Formula # 5367-45-34
Serving size 55 g, 5 g alginate

| # | Ingredient | Percentage |
|---|---|---|
| 1 | Sugar-Free Choc Coating | 20.00 |
| 2 | Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 5.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

Chocolate Peanut Butter
Formula # 5367-45-35
Serving size 55 g, 3 g alginate

| # | Ingredient | Percentage |
|---|---|---|
| 1 | Sugar-Free Choc Coating | 20.00 |
| 2 | High Fructose Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| 7 | Inulin | 5.00 |
| 8 | Erythritol | 5.00 |
| 9 | Alginate | 5.45 |
| 10 | Peanut Flour | 3.40 |
| 11 | Dicalcium Phosphate Anyhdrous | 1.88 |
| 12 | Soy Protein Isolate | 4.00 |
| 13 | Calcium Caseinate | 4.00 |
| 14 | Whey Protein Isolate | 4.00 |
| 15 | Peanuts | 9.27 |
| | Total | 100.00 |

Chocolate Peanut Butter
Formula # 5367-45-01
Serving size 55 g, contains 200 mg Ca, 1 g alginate, 1 g pectin, 3 g inulin

| # | Ingredient | Percentage |
|---|---|---|
| 1 | Sugar-Free Choc Coating | 18.00 |
| 2 | Water | 12.50 |
| 3 | Glycerine | 9.00 |
| 4 | Peanut Butter | 10.00 |
| 8 | Peanut Butter Flavor | 1.00 |
| 11 | Inulin | 5.45 |
| 12 | Erythritol | 8.00 |
| 13 | Fructose | 3.50 |
| 15 | Alginate | 2.15 |
| 16 | Pectin | 3.04 |
| 17 | Soy Protein Isolate | 5.50 |
| 18 | Calcium Caseinate | 5.50 |
| 19 | Whey Protein Isolate | 8.00 |
| 20 | Tricalcium Phosphate | 0.96 |
| 21 | Peanut Flour | 7.40 |
| | Total | 100.00 |
| | Aw 0.686 | |

Chocolate Peanut Butter
Formula # 5367-45-02
Serving size 55 g, contains 200 mg Ca, 1 g alginate,
1 g pectin, 3 g inulin

| # | Ingredient | Percentage |
|---|---|---|
| 1 | Sugar-Free Choc Coating | 15.00 |
| 2 | Water | 14.00 |
| 3 | Glycerine | 10.00 |
| 4 | Peanut Butter | 10.00 |
| 8 | Peanut Butter Flavor | 2.00 |
| 11 | Inulin | 5.45 |
| 12 | Erythritol | 7.00 |
| 15 | Alginate | 2.15 |
| 16 | Pectin | 3.04 |
| 21 | Peanut Flour | 5.00 |
| 20 | Tricalcium Phosphate | 0.96 |
| 17 | Soy Protein Isolate | 5.00 |
| 18 | Calcium Caseinate | 5.00 |
| 19 | Whey Protein Isolate | 7.50 |
| | Peanuts | 5.00 |
| | Soy Crisps | 2.90 |
| | Total | 100.00 |

Aw 0.726

Chocolate Peanut Butter
Formula # 5367-45-03
Serving size 55 g, contains 200 mg Ca, 1 g alginate,
1 g pectin, 3 g inulin

| # | Ingredient | Percentage |
|---|---|---|
| 1 | Sugar-Free Choc Coating | 15.00 |
| 2 | Water | 14.50 |
| 3 | Glycerine | 11.00 |
| 4 | Peanut Butter | 10.00 |
| 8 | Peanut Butter Flavor | 2.00 |
| 11 | Inulin | 5.45 |
| 12 | Erythritol | 10.00 |
| 15 | Alginate | 2.15 |
| 16 | Pectin | 3.04 |
| 21 | Peanut Flour | 3.40 |
| 20 | Tricalcium Phosphate | 0.96 |
| 17 | Soy Protein Isolate | 3.50 |
| 18 | Calcium Caseinate | 3.50 |
| 19 | Whey Protein Isolate | 3.50 |
| | Hydrolysed Whey | 3.50 |
| | Peanuts | 6.00 |
| | Soy Crisps | 2.50 |
| | Total | 100.00 |

Aw 0.710

Chocolate Raspberry
Formula # 5367-44-01
Serving size 45 g, contains 200 mg Ca, 5% alginate/pectin mix,
3 g inulin

| # | Ingredient | Percentage |
|---|---|---|
| 1 | Chocolate Coating | 15.00 |
| 2 | HFCS | 10.00 |
| 3 | Water | 10.00 |
| 4 | Glycerine | 8.00 |
| 5 | Honey | 2.00 |
| 6 | Canola Oil | 6.00 |
| 7 | Raspberry Flavor | 1.00 |
| 8 | Raspberry Flavor | 0.50 |
| 9 | White Chocolate Flavor | 0.35 |
| 10 | Vanilla Flavor | 0.20 |
| 11 | Inulin | 6.70 |
| 12 | Erythritol | 5.00 |
| 13 | Malic Acid | 0.25 |
| 14 | Tricalcium Phosphate | 1.15 |
| 15 | Alginate Pectin Mix | 5.00 |
| 16 | Soy Protein Isolate | 7.00 |
| 17 | Calcium Caseinate | 6.00 |
| 18 | Whey Protein Isolate | 6.00 |
| 19 | Dried Raspberry | 4.85 |
| 20 | Soy Crisps, 80% protein | 5.00 |
| | Total | 100.00 |

Aw at 0.677

Chocolate Peanut Butter
Formula # 5367-45-04
Serving size 55 g, contains 200 mg Ca, 1 g alginate,
1 g pectin, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 6.00 |
| 8 | Alginate | 2.15 |
| 9 | Pectin | 3.04 |
| 10 | Peanut Flour | 3.40 |
| 11 | Tricalcium Phosphate | 0.96 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Total | 100.00 |

Aw 0.698

Chocolate Raspberry
Formula # 5367-44-02
Serving size 45 g, contains 200 mg Ca,
5% alginate/pectin mix, 3 g inulin

| # | Ingredient | Percentage |
|---|---|---|
| 1 | Chocolate Coating | 15.00 |
| 2 | HFCS | 10.50 |
| 3 | Water | 10.50 |
| 4 | Glycerine | 8.00 |
| 5 | Honey | 2.00 |
| 6 | Canola Oil | 6.00 |
| 7 | Raspberry Flavor | 1.00 |
| 8 | Raspberry Flavor | 0.50 |
| 9 | White Chocolate Flavor | 0.35 |
| 10 | Vanilla Flavor | 0.50 |
| 11 | Inulin | 6.70 |
| 12 | Erythritol | 2.00 |
| 13 | Malic Acid | 0.25 |
| 14 | Alginate | 2.50 |
| 15 | Pectin | 2.50 |
| 16 | Soy Protein Isolate | 7.00 |
| 17 | Calcium Caseinate | 6.00 |
| 18 | Whey Protein Isolate | 6.00 |
| 19 | Tricalcium Phosphate | 1.15 |
| 20 | Dried Raspberry | 6.00 |
| 21 | Soy Crisps, 80% protein | 5.55 |
| | Total | 100.00 |

Chocolate Peanut Butter
Formula # 5367-45-31
Serving size 55 g, 5 g alginate

| # | Ingredient | Percentage |
|---|---|---|
| 1 | Sugar-Free Choc Coating | 18.20 |
| 2 | HFCS | 12.00 |
| 3 | Maltitol | 12.00 |
| 4 | Glycerine | 3.00 |
| 5 | Peanut Butter | 3.00 |
| 6 | Peanut Butter Flavor | 3.00 |
| 7 | Vanilla | 0.50 |
| | Mix for 0.5 min | |
| 8 | Alginate | 9.10 |
| | Mix for 1 min | |
| 9 | Erythritol | 8.00 |
| 10 | Inulin | 5.00 |
| 11 | Peanut Flour | 5.00 |
| 12 | Hydrolyzed Whey Isolate | 4.00 |
| 13 | Sucrolose | 0.01 |
| | Mix for 0.5 min | |
| 14 | Tricalcium Phosphate | 0.00 |
| 15 | Calcium Carbonate | 0.00 |
| 16 | Whey Protein Isolate | 8.50 |
| 17 | Peanuts | 8.69 |
| | Mix for 1 min | |
| | Total | 100.00 |

Aw = 0.52

Chocolate Peanut Butter
Formula # 5367-45-05
Serving size 55 g, contains 200 mg Ca, 1 g alginate,
1 g pectin, 3 g inulin
GHB

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 6.00 |
| 8 | Alginate | 4.30 |
| 9 | Pectin | 0.00 |
| 10 | Peanut Flour | 3.40 |
| 11 | Tricalcium Phosphate | 0.96 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Total | 99.11 |

Aw 0.713

Chocolate Peanut Butter
Formula # 5367-45-06
Serving size 55 g, contains 200 mg Ca, 1 g alginate,
1 g pectin, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 7.00 |
| 7 | Erythritol | 6.00 |
| 8 | Alginate | 0.00 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | MonoCalcium Phosphate | 4.60 |
| | Total | 100.00 |

a = 0.705

Chocolate Peanut Butter
Formula # 5367-45-07
Serving size 55 g, contains 200 mg Ca, 1 g alginate,
1 g pectin, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 6.10 |
| 7 | Erythritol | 6.00 |
| 8 | Alginate | 0.00 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 5.50 |
| | Total | 100.00 |

Aw 0.690

Chocolate Peanut Butter, Dec. 22, 2003
Formula # 5367-45-08
Serving size 55 g, contains 300 mg Ca, 1 g alginate,
1 g pectin, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 2.76 |
| 8 | Alginate | 2.15 |
| 9 | Pectin | 3.04 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 4.20 |
| | Total | 100.00 |

Chocolate Peanut Butter
Formula # 5367-45-09
Serving size 55 g, contains 300 mg Ca, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 5.52 |
| 8 | Alginate | 0.00 |
| 9 | Pectin | 0.00 |
| 10 | Peanut Flour | 8.59 |
| 11 | Tricalcium Phosphate | 1.44 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| 18 | Calcium Lactate | 0.00 |
| | Total | 100.00 |

Chocolate Peanut Butter
Formula # 5367-45-10
Serving size 55 g, contains 200 mg Ca, 3 g inulin
Test layer bar

| # | Ingredient | Percentage |
|---|---|---|
| 1 | Sugar-Free Choc Coating | 15.00 |
| | Caramel | 13.62 |
| 8 | Alginate | 4.30 |
| 9 | Pectin | 6.08 |
| | Water | 10.00 |
| | Glycerine | 10.00 |
| 16 | Peanuts | 6.00 |
| 2 | Water | 4.00 |
| 3 | Glycerine | 3.00 |
| 4 | Peanut Butter | 4.00 |
| 5 | Peanut Butter Flavor | 1.00 |
| 6 | Inulin | 15.57 |
| 10 | Peanut Flour | 2.00 |
| 11 | Tricalcium Phosphate | 2.74 |
| 13 | Calcium Caseinate | 2.69 |
| | Total | 100.00 |

Chocolate Peanut Butter
Formula # 5367-45-11
Serving size 55 g
Test layer bar

| # | Ingredient | Percentage |
|---|---|---|
| 1 | Sugar-Free Choc Coating | 15.00 |
| | Caramel | 13.62 |
| 8 | Alginate | 4.30 |
| 9 | Pectin | 6.08 |
| | Water | 0.00 |
| | Glycerine | 10.00 |
| 16 | Peanuts | 6.00 |
| 2 | Water | 4.00 |
| 3 | Glycerine | 3.00 |
| 4 | Peanut Butter | 4.00 |
| 5 | Peanut Butter Flavor | 1.00 |
| 6 | Inulin | 15.57 |
| 10 | Peanut Flour | 2.00 |
| 11 | Tricalcium Phosphate | 2.74 |
| 13 | Calcium Caseinate | 2.69 |
| | Total | 90.00 |

Chocolate Peanut Butter
Formula # 5367-45-12
Serving size 55 g, contains 300 mg Ca

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 2.76 |
| 8 | Alginate | 5.19 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 4.20 |
| | Total | 100.00 |

### Example 3

A study to evaluate the effects of soluble fiber and soluble calcium on food intake was performed by the following procedure.

The study was a within-subjects design with 30 participants completing three one week treatment periods, with a washout period of one week between treatment periods. Treatment order was counterbalanced to have five subjects randomly assigned to each of six possible treatment sequences. Subjects in each treatment period consumed a test beverage at breakfast and after lunch (mid-afternoon). In one treatment period, subjects consumed a placebo beverage without fiber. In two treatment periods, the test beverage contained a blend of soluble fibers of one of the following compositions:

| | 2.8 g Fiber | 11.0 g Fiber | Placebo |
|---|---|---|---|
| Ingredient | % | % | % |
| Water | 95.470 | 96.400 | 97.010 |
| Trisodium citrate dihydrate | 0.250 | 0.250 | 0.250 |
| LBA alginate (ISP) | 0.640 | 0.210 | 0.000 |
| GHB alginate (ISP) | 0.550 | 0.180 | 0.000 |
| USP L200 pectin (Kelco) | 0.200 | 0.066 | 0.000 |
| Apple juice concentrate | 2.300 | 2.300 | 2.300 |
| EDTA | 0.002 | 0.002 | 0.002 |
| Sucralose | 0.011 | 0.011 | 0.011 |
| Malic acid, granular | 0.200 | 0.200 | 0.200 |
| Red 40, 10% solution | 0.001 | 0.001 | 0.001 |
| Flavor | 0.380 | 0.380 | 0.380 |
| Total | 100.000 | 100.0001 | 100.000 |

The fiber drinks were consumed with a separate beverage containing calcium lactate (not more than 500 mg elemental calcium per serving). The placebo was taken with a second placebo beverage matched for flavor and calories, but without calcium lactate. The test drink containing calcium lactate or corresponding placebo had the following composition:

| | Calcium Placebo | Calcium Free Placebo |
|---|---|---|
| Ingredient | % | % |
| Water | 96.430 | 99.846 |
| Calcium lactate | 3.065 | 0.000 |
| Malic acid | 0.330 | 0.330 |
| Sucralose | 0.050 | 0.020 |
| Yellow #5, 1% solution | 0.007 | 0.007 |
| Red #40, 1% Solution | 0.0069 | 0.0069 |
| Flavor | 0.110 | 0.110 |
| Total | 100.000 | 100.000 |

Subjects in the study were premenopausal women selected without regard to racial or ethnic background. Eligible women were between 20 and 40 years of age, non-smokers, and overweight or obese (body mass index, or BMI, of 25-35 kg per square meter). Test Sessions and Experimental Measurements

Test sessions occurred on the first and seventh day of the use of each experimental period. The night before the sessions, subjects consumed an evening meal of their own choosing that was replicated the night before each test session. Test sessions began between 7:00 and 9:00 AM. Subjects first completed a short questionnaire to ensure they consumed the evening meal, and had not been ill in the previous week. Immediately before a standardized breakfast meal (choice of bagel or raisin bran cereal) they were asked to consume a fiber test beverage within a three minute interval, which included the first part of the test beverage (fiber or placebo) first, immediately followed by the second part of the test beverage (calcium or placebo). They were then served the standard breakfast. They returned to the lab for lunch 4-5 hours later, and dinner 9-10 hours later. They were provided with a portable cooler containing the test beverage (fiber or placebo beverage), and the calcium beverage or calcium-free placebo beverage), and a bottle of water. They were instructed to consume the test beverage 2½ hours after the completion of lunch and not to consume any food during the day except the test meals provided, the test beverages, and the bottled water.

At the test sessions, lunch and dinner were provided as buffet-style meals. Subjects were also provided snacks for consumption during the evening. They were told to consume as much of the snacks as they desired. Lunch and dinner servings of each individual food were weighed to the nearest 0.1 g before and after consumption to determine caloric and macronutrient intake. Evening snacks were returned to the test site to determine food consumption.

Subjects were asked to consume 14 test drinks during each week of the three week long experimental periods. On Day 1, as mentioned above, they drank one two-part test beverage before breakfast and one 2.5 hours after lunch. Additionally, on the first test day they were provided with five refrigerated test beverages (5 first part and 5 second part) to take home. They were instructed to consume one test beverage, which is one first part followed by one second part, before breakfast, and another test beverage about 2½ hours after lunch each day on the second through sixth days. Subjects returned to the laboratory on the seventh day to repeat the procedure of the first day.

Data Analysis

Data were analyzed using the Statistical Analysis System (SAS Version 8.2, Cary, N.C.). The mixed model procedure is used to test for treatment differences, with treatment condition (low fiber, high fiber, and placebo), day (1 or 7) and the interaction of condition and day entered into the statistical models. The effects of treatment session was also tested as a covariate and kept in the final model when found to be significant. The endpoint measurements included the total daily energy and macronutrient content of foods consumed, as well as at each individual meal (breakfast, lunch, dinner, and evening snack).

Consumption of the two different fiber containing beverages (1 g and 2.8 g per serving) resulted in a trend toward reduction in total calorie intake measured over the 24 hour period beginning with the morning beverage.

Effect of Fiber Beverages on Total Calorie

| | Mean | | |
|---|---|---|---|
| Condition | Kcal Intake | Standard Error | P value vs. placebo |
| Placebo | 2634 | 109 | 0.17 |
| 1 g fiber beverage | 2512 | 110 | 0.17 |
| 2.8 g fiber beverage | 2510 | 109 | |

Consumption of both the fiber containing beverages (1 g and 2.8 g per serving) resulted in a significant decrease in food consumption at dinner, as shown below. Effect of Fiber Beverages on Caloric Intake at Dinner

| | Mean | | |
|---|---|---|---|
| Condition | Kcal Intake | Standard Error | P value vs. placebo |
| Placebo | 765 | 37 | |
| 1 g fiber beverage | 689 | 37 | 0.039 |
| 2.8 g fiber beverage | 678 | 37 | 0.016 |

The 1 g fiber beverage reduced dinner food intake by an average of 76 kcal, and the 2.8 g beverage provided a reduction of 87 kcal. The P values, determined by a post-hoc Tukey's analysis, indicated that these results were statistically significant (p<0.05).

Further analysis of the nutrient composition of the individual foods consumed indicated that the consumption of the fiber beverages was associated with a significant reduction in the intake of carbohydrates at dinner, as shown below. Effect of Fiber Beverages on Carbohydrate Caloric Intake at Dinner

| | Mean Carbohydrate | Standard | P value |
|---|---|---|---|
| Condition | Kcal Intake | Error | vs. placebo |
| Placebo | 379 | 21 | |
| 1 g fiber beverage | 329 | 21 | 0.007 |
| 2.8 g fiber beverage | 324 | 21 | 0.003 |

The 1 g beverage reduced carbohydrate intake at dinner by 50 kcal, and the 2.8 g beverage provided a 55 kcal reduction. The reduction in carbohydrate intake at both levels was statistically significant (p<0.01).

The fiber beverages also reduced total daily food intake, as shown below.

Effects of Fiber Beverages on Daily Caloric Intake

| | Mean | | |
|---|---|---|---|
| Condition | Kcal Intake | Standard Error | P value vs. placebo |
| Placebo | 1353 | 64 | |
| 1 g fiber beverage | 1261 | 64 | 0.026 |
| 2.8 g fiber beverage | 1264 | 64 | 0.033 |

The 1 g fiber beverage reduced overall food intake on the test day by an average of 92 kcal, and the 2.8 g beverage provided a reduction of 89 kcal. The P values, determined by a post-hoc Tukey's analysis, indicated that these results were statistically significant (p<0.05). These results indicated the absence of compensatory eating that could have occurred in response to the reduced dinner caloric intake.

### Example 4

### Crispy Formulations

A variety of crispy formulations were prepared using the formulations as shown below followed by extrusion to make crispies:

To produce a batch of crispies, the ingredients were dry blended in a small ribbon blender. The resulting dry blend was transferred using a feeder, e.g., a K-Tron loss-in-weight feeder, into the hopper of an extruder, e.g., a Buhler Twin Screw Extruder configured with at least one heating unit, e.g., two Mokon barrel-heating units. Water was added as steam to the dry blend using a two barrel injection system for adding water and a second liquid into the barrel at variable rates. The blend was then mixed and cooked in the extruder. The hot pressurized product stream was forced through a die for expansion and then conveyed by vacuum to a fluid bed drier, e.g., Buhler fluid bed drier, and dried to the desired moisture content. The fluid bed drier dried about 50 to about 100 kg/hour at temperatures from about 20 to about 110° C.

Batch 1A - 5367-54-01A

| | Ingredients | % |
|---|---|---|
| 1 | Rice Flour | 52.30 |
| 2 | Alginate LBA | 25.20 |
| 3 | Whey Protein Isolate | 20.00 |
| 4 | Starch | 2.00 |
| 5 | Salt | 0.50 |
| | Total | 100.00 |

Batch 2, 5367-54-02

| | Ingredients | % |
|---|---|---|
| 1 | Rice Flour | 54.30 |
| 2 | Alginate LBA | 25.20 |
| 3 | Whey Protein Isolate | 12.00 |
| 4 | Starch | 8.00 |
| 5 | Salt | 0.50 |
| | Total | 100.00 |

Batch 3, 5367-54-03

| | Ingredients | % |
|---|---|---|
| 1 | Rice Flour | 66.30 |
| 2 | Alginate LBA | 25.20 |
| 3 | Whey Protein Isolate | 00.00 |
| 4 | Starch | 8.00 |
| 5 | Salt | 0.50 |
| | Total | 100.00 |

Batch 4, 5367-54-04

| | Ingredients | % |
|---|---|---|
| 1 | Rice Flour | 54.30 |
| 2 | Alginate LBA | 12.00 |
| 3 | Alginate DPB | 6.00 |
| 4 | Alginate KTHV | 7.20 |
| 5 | Whey Protein Isolate | 12.00 |
| 6 | Starch | 8.00 |
| 7 | Salt | 0.50 |
| | Total | 100.00 |

Batch #1, repeat 5367-54-04

| | Ingredients | % |
|---|---|---|
| 1 | Rice Flour | 54.30 |
| 2 | Alginate LBA | 12.00 |
| 3 | Alginate DPB | 6.00 |
| 4 | Alginate KTHV | 7.20 |
| 5 | Whey Protein Isolate BiPro | 12.00 |
| 6 | Wheat Starch | 8.00 |
| 7 | Salt | 0.50 |
| | Total | 100.00 |

Batch #2, based on 5367-54-04
Formula # 5981-04-01

| | Ingredients | % |
|---|---|---|
| 1 | Rice Flour | 54.30 |
| 2 | Alginate DPB | 25.20 |
| 3 | Whey Protein Isolate BiPro | 12.00 |
| 4 | Whey Starch | 8.00 |
| 5 | Salt | 0.50 |
| | Total | 100.00 |

Batch #3, Same as Batch #2 (5981-04-01),
replacing DPB with KTHV
Formula # 5981-04-02

| | Ingredients | % |
|---|---|---|
| 1 | Rice Flour | 54.30 |
| 2 | Alginate KTHV | 25.20 |
| 3 | Whey Protein Isolate BiPro | 12.00 |
| 4 | Whey Starch | 8.00 |
| 5 | Salt | 0.50 |
| | Total | 100.00 |

Batch #4: Based on 5981-04-01, increased alginate by 25%
Formula # 5981-04-03

| | Ingredients | % |
|---|---|---|
| 1 | Rice Flour | 56.00 |
| 2 | Alginate KTHV | 31.50 |
| 3 | Whey Protein Isolate BiPro | 8.00 |
| 4 | Corn Starch | 4.00 |
| 5 | Salt | 0.50 |
| | Total | 100.00 |

Batch #5: Based on 5981-04-01, increased alginate by 50%
Formula # 5981-04-04

| | Ingredients | % |
|---|---|---|
| 1 | Rice Flour | 49.70 |
| 2 | Alginate KTHV | 37.80 |
| 3 | Whey Protein Isolate BiPro | 8.00 |
| 4 | Corn Starch | 4.00 |
| 5 | Salt | 0.50 |
| | Total | 100.00 |

Formula # 5981-04-05

| | Ingredients | % |
|---|---|---|
| 1 | Rice Flour | 46.00 |
| 2 | Alginate KTHV | 31.50 |
| 3 | Whey Protein Isolate BiPro | 8.00 |
| 4 | Corn Starch | 4.00 |
| 5 | Salt | 0.50 |
| 6 | Inulin | 10.00 |
| | Total | 100.00 |

Formula # 5981-04-06

| | Ingredients | % |
|---|---|---|
| 1 | Corn Meal | 56.00 |
| 2 | Alginate KTHV | 31.50 |
| 3 | Whey Protein Isolate BiPro | 8.00 |
| 4 | Corn Starch | 4.00 |
| 5 | Salt | 0.50 |
| | Total | 100.00 |

Formula # 5981-04-07

| | Ingredients | % |
|---|---|---|
| 1 | Corn Cone | 64.00 |
| 2 | Alginate KTHV | 31.50 |
| 3 | Whey Protein Isolate BiPro | 8.00 |
| 4 | Corn Starch | 0.00 |
| 5 | Salt | 0.50 |
| 6 | Inulin F97 | 0.00 |
| | Total | 100.00 |

Formula # 5981-04-08

| | Ingredients | % |
|---|---|---|
| 1 | Corn Cone | 53.70 |
| 2 | Alginate KTHV | 37.80 |
| 3 | Whey Protein Isolate BiPro | 0.00 |
| 4 | Corn Starch | 0.00 |
| 5 | Salt | 0.50 |
| 6 | Inulin F97 | 8.00 |
| | Total | 100.00 |

Batch #10
Formula # 5981-04-09

| | Ingredients | % |
|---|---|---|
| 1 | Corn Cone | 46.70 |
| 2 | Alginate KTHV | 42.80 |
| 3 | Whey Protein Isolate BiPro | 0.00 |
| 4 | Corn Starch | 0.00 |
| 5 | Salt | 0.50 |
| 6 | Inulin F97 | 10.00 |
| | Total | 100.00 |

Batch #11
Formula # 5981-04-10

| | Ingredients | % |
|---|---|---|
| 1 | Corn Cone | 49.50 |
| 2 | Alginate KTHV | 50.00 |
| 3 | Whey Protein Isolate BiPro | 0.00 |
| 4 | Corn Starch | 0.00 |
| 5 | Salt | 0.50 |
| 6 | Inulin F97 | 0.00 |
| | Total | 100.00 |

Batch #12, same formula as 5981-04-07, used different die to make curls
Formula # 5981-04-11

| | Ingredients | % |
|---|---|---|
| 1 | Corn Cone | 56.00 |
| 2 | Alginate KTHV | 31.50 |
| 3 | Whey Protein Isolate BiPro | 4.00 |
| 4 | Corn Starch | 0.00 |
| 5 | Salt | 0.50 |
| 6 | Inulin F97 | 8.00 |
| | Total | 100.00 |

Batch #13 = batch #10 5981-04-09
Formula # 5981-04-12

| | Ingredients | % |
|---|---|---|
| 1 | Corn Cone | 46.70 |
| 2 | Alginate KTHV | 42.80 |
| 3 | Whey Protein Isolate BiPro | 0.00 |
| 4 | Corn Starch | 0.00 |
| 5 | Salt | 0.50 |
| 6 | Inulin F97 | 10.00 |
| | Total | 100.00 |

Batch #14: same as batch #4, 5981-04-03 except replaced KTHV with
DPB
Formula # 5981-15-01

| | Ingredients | % |
|---|---|---|
| 1 | Rice Flour | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #15: based on batch #14
Formula # 5981-15-02

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Cocoa | 5.00 |
| | Total | 100.00 |

Batch #16: based on batch #14
Formula # 5981-15-03

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 4.00 |
| 5 | Salt | 0.50 |
| 6 | Micro-crystalline Cellolose | 4.00 |
| | Total | 100.00 |

Batch #17: based on batch 14
Formula # 5981-15-04

| | Ingredients | % |
|---|---|---|
| 1 | Rice Flour | 56.00 |
| 2 | Alginate DPB | 31.50 |
| 3 | Whey Protein Isolate BiPro | 7.00 |
| 4 | Corn Starch | 4.00 |
| 5 | Salt | 0.50 |
| 6 | Glycerine | 1.00 |
| | Total | 100.00 |

Batch #18: based on batch # 14
Formula # 5981-15-05

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Oil | 5.00 |
| | Total | 100.00 |

Batch #19: based on batch # 14
Formula # 5981-15-06

| | Ingredients | % |
|---|---|---|
| | Rice Flour | 56.00 |
| 2 | Alginate DPB | 31.50 |
| 3 | Whey Protein Isolate BiPro | 7.95 |
| 4 | Corn Starch | 4.00 |
| 5 | Salt | 0.50 |
| 6 | Sucrolose | 0.05 |
| | Total | 100.00 |

Batch #20: based on batch # 14
Formula # 5981-15-07

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Trehalose | 5.00 |
| | Total | 100.00 |

Batch #21: based on batch # 7, 5981-04-06
Formula # 5981-15-08

| | Ingredients | % |
|---|---|---|
| 1 | Corn Cone | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #22: same as batch # 21
Formula # 5981-15-09

| | Ingredients | % |
|---|---|---|
| 1 | Corn Cone 2 | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #25
Formula # 5981-15-12

| | Ingredients | % |
|---|---|---|
| 1 | Corn Cone | 54.00 |
| 2 | Alginate DPB | 31.50 |
| 3 | Whey Protein Isolate BiPro | 2.00 |
| 4 | Corn Starch | 2.00 |
| 5 | Salt | 0.50 |
| 6 | Corn Fiber | 10.00 |
| | Total | 100.00 |

Formula # 5981-15-15

| | Ingredients | % |
|---|---|---|
| 1 | Rice Flour | 56.50 |
| 2 | Alginate DPB | 31.50 |
| 3 | Whey Protein Isolate BiPro | 4.00 |
| 4 | Corn Starch | 3.00 |
| 5 | Fractionated Canola Oil | 5.00 |
| | Total | 100.00 |

Formula # 5981-15-25

| | Ingredients | % |
|---|---|---|
| 1 | Rice Flour | 88.00 |
| 2 | Whey Protein Isolate BiPro | 4.00 |
| 3 | Corn Starch | 3.00 |
| 4 | Fractionated Canola Oil | 5.00 |
| | Total | 100.00 |

### Example 5—Bars with Crispies

A variety of bar formulations incorporating various crispies formulations set forth above were prepared as shown below:

Formula 5367-52-01

| # | Ingredients | % in Bar |
|---|---|---|
| 1 | High Maltose Corn Syrup | 15.36 |
| 2 | HFCS | 4.80 |
| 3 | Molasses | 0.64 |
| 4 | Honey | 0.64 |
| 5 | Granulated Sugar | 4.16 |
| 6 | Salt | 0.32 |
| 7 | Citric Acid | 0.06 |

Step 1: Mix well, cook to brix at 88.5%

| | | |
|---|---|---|
| 8 | Erythritol | 2.18 |
| 9 | Inulin | 0.00 |
| 10 | Calcium Carbonate | 1.60 |

Step 2: Add dry ingredients slowly to syrup above,
mix well using high shear mixer

| | | |
|---|---|---|
| 11 | Canola Oil | 1.28 |
| 12 | Lecithin | 0.32 |
| 13 | Vanilla Flavor | 0.48 |
| 14 | Cranberry Flavor | 0.16 |

Step 3, Add rest of liquid ingredients, mix well.
Using high shear mixer

| | | |
|---|---|---|
| | Total | 32.00 |
| 15 | Alginate Crisps (5367-54-00) | 38.00 |
| 16 | Oats | 10.0 |
| 17 | Whole Almond | 8.0 |
| 18 | Raisins | 6.0 |
| 19 | Sweetened Cranberry | 6.0 |

Step 4: Add binder to dry ingredients, mix well quickly
Step 5: Add the mass to a pan, roll to right density, cool
at refrigerator for 20 mm, cut to desired size

| | |
|---|---|
| Total | 100.00 |

Formula 5367-52-02
Based on 5367-52-01, replaced calcium carbonate with inulin

| # | Ingredients | % in Bar |
|---|---|---|
| 1 | High Maltose Corn Syrup | 15.36 |
| 2 | HFCS | 4.80 |
| 3 | Molasses | 0.64 |
| 4 | Honey | 0.64 |
| 5 | Granulated Sugar | 4.16 |
| 6 | Salt | 0.32 |
| 7 | Citric Acid | 0.06 |

Step 1: Mix well, cook to brix at 88.5%

| | | |
|---|---|---|
| 8 | Erythritol | 2.18 |
| 9 | Inulin | 1.60 |
| 10 | Calcium Carbonate | 0.00 |

Step 2: Add dry ingredients slowly to syrup above,
mix well using high shear mixer

| | | |
|---|---|---|
| 11 | Canola Oil | 1.28 |
| 12 | Lecithin | 0.32 |
| 13 | Vanilla Flavor | 0.48 |
| 14 | Cranberry Flavor | 0.16 |

Step 3, Add rest of liquid ingredients, mix well. Using high shear mixer

| | | |
|---|---|---|
| | Total | 32.00 |
| 15 | Alginate Crisps (5367-54-00) | 38.00 |
| 16 | Oats | 10.0 |
| 17 | Whole Almond | 8.0 |
| 18 | Raisins | 6.0 |
| 19 | Sweetened Cranberry | 6.0 |

Step 4: Add binder to dry ingredients, mix well quickly
Step 5: Add the mass to a pan, roll to right density, cool at refrigerator
for 20 min, cut to desired size

| | |
|---|---|
| Total | 100.00 |

Formula 5981-07-01

| # | Ingredients | % in Bar |
|---|---|---|
| 1 | High Maltose Corn Syrup | 15.36 |
| 2 | HFCS | 4.80 |
| 3 | Granulated Sugar | 3.84 |
| 4 | Erythritol | 3.14 |
| 5 | Fructose | 1.92 |
| 6 | Canola Oil | 0.64 |
| 7 | Molasses | 0.48 |
| 8 | Honey | 0.48 |
| 9 | Salt | 0.48 |
| 10 | Vanilla Flavor | 0.48 |
| 11 | Lecithin | 0.16 |
| 12 | Cranberry Flavor | 0.16 |
| 13 | Citric Acid | 0.06 |
| 14 | Calcium Carbonate | 0.00 |
| | Total | 32.00 |

Step 1: Mix all ingredients except flavors, oil and lecithin,
cook at 180 to 200 Brix 88%
Step 2: Add flavors and oil and lecithin, check Brix to 87%

| | | |
|---|---|---|
| 15 | Alginate Crisps (Batch #4, 5981-04-03) | 39.7 |
| 16 | Oats | 8.0 |
| 17 | Whole Almond | 7.3 |
| 18 | Raisins | 7.0 |
| 19 | Sweetened Cranberry | 6.0 |

Step 3, Add 640 g syrup to dry ingredients, mix well quickly
Step 4: Transfer mass to a pan, roll it flat, cool down in
refrigerator for minimum 15 min
Step 5: Cut to L 3.5″, W 1.2″ and H 0.8″, wrap them

| | |
|---|---|
| Total | 100.00 |

Results:
1) Good tasting bar, loosely bound, not sticky.

Formula 5981-07-02

| # | Ingredients | % in Bar |
|---|---|---|
| 1 | High Maltose Corn Syrup | 15.36 |
| 2 | HFCS | 4.80 |
| 3 | Granulated Sugar | 3.36 |
| 4 | Fructose | 1.92 |
| 5 | Canola Oil | 0.64 |
| 6 | Molasses | 0.48 |
| 7 | Honey | 0.48 |
| 8 | Salt | 0.48 |
| 9 | Vanilla Flavor | 0.48 |
| 10 | Lecithin | 0.16 |
| 11 | Cranberry Flavor | 0.16 |
| 12 | Citric Acid | 0.06 |
| 13 | Monocalcium Phosphate, | 3.62 |
| | Monohydrate . . . | |
| | Total | 32.00 |

Step 1: Mix all ingredients except flavors, oil and lecithin,
cook at 180 to 200 Brix 88%
Step 2: Add flavors, oil and lecithin, mix well, check Brix to 87%

| | | |
|---|---|---|
| 14 | Alginate Crisps (Batch #4, 5981-04-03) | 45.4 |
| 15 | Oats | 0 |
| 16 | Whole Almond | 7.6 |
| 17 | Raisins | 7.0 |
| 18 | Sweetened Cranberry | 8.0 |

Step 3, Add 640 g syrup to dry ingredients, mix quickly
Step 4: Transfer the mass to a pan, roll it flat, cool down in
refrigerator for minimum 15 min
Step 5: Cut to L 3.5″, W 1.2″ and H 0.8″, then wrap

| | |
|---|---|
| Total | 100.00 |

Formula 5981-07-03

| # | Ingredients | % in Bar |
|---|---|---|
| 1 | High Maltose Corn Syrup | 15.36 |
| 2 | HFCS | 5.34 |
| 3 | Granulated Sugar | 3.36 |
| 4 | Fructose | 1.92 |
| 5 | Canola Oil | 0.64 |
| 6 | Molasses | 0.48 |
| 7 | Honey | 0.48 |
| 8 | Salt | 0.48 |
| 9 | Vanilla Flavor | 0.48 |
| 10 | Lecithin | 0.16 |
| 11 | Cranberry Flavor | 0.16 |
| 12 | Citric Acid | 0.06 |
| 13 | Dicalcium Phosphate Dihydrate | 3.07 |
| | Total | 32.00 |

Step 1: Mix all ingredients except flavors, oil and lecithin,
cook at 180 to 200 Brix 88%
Step 2: Add flavors, oil and lecithin, mix well, check Brix to 87%

| | | |
|---|---|---|
| 15 | Alginate Crisps (Batch #4, 5981-04-03) | 45.4 |
| 16 | Oats | 0 |
| 17 | Whole Almond | 7.6 |
| 18 | Raisins | 7.0 |
| 19 | Sweetened Cranberry | 8.0 |

Step 3, Add 640 g syrup to dry ingredients, mix quickly
Step 4: Transfer the mass to a pan, roll it flat, cool down in
refrigerator for minimum 15 min
Step 5: Cut to L 3.5″, W 1.2″ and H 0.8″, then wrap

| | |
|---|---|
| Total | 100.00 |

Formula 5981-07-04

| # | Ingredients | % in Bar |
|---|---|---|
| 1 | High Maltose Corn Syrup | 16.32 |
| 2 | HFCS | 5.81 |
| 3 | Granulated Sugar | 2.38 |
| 4 | Fructose | 2.04 |
| 5 | Molasses | 0.68 |
| 6 | Honey | 0.51 |
| 7 | Salt | 0.51 |
| 8 | Dicalcium Phosphate Dihydrate | 5.00 |

Step 1: Weigh and cook above ingredients at 180 to 88% Brix

| | | |
|---|---|---|
| 9 | Vanilla Flavor | 0.51 |
| 10 | Cranberry Flavor | 0.17 |
| 11 | Citric Acid | 0.07 |
| | Total | 34.00 |

Step 2: Add flavors, mix well, and cook gently, check Brix to 87%

| | | |
|---|---|---|
| 12 | Alginate Crisps (Batch #4, 5981-04-03) | 32.0 |
| 13 | Rolled Oats | 14.0 |
| 14 | Whole Almond | 6.0 |
| 15 | Raisins | 7.0 |
| 16 | Sweetened Cranberry | 8.0 |

Step 3, Add 680 g syrup to dry ingredients, mix quickly
Step 4: Transfer the mass to a pan, roll it flat, cool down in
refrigerator for minimum 15 min
Step 5: Cut to L 3.5″, W 1.2″ and H 0.8″, then wrap

| | |
|---|---|
| Total | 100.00 |

Formula 5981-07-06

| # | Ingredients | % in Bar |
|---|---|---|
| 1 | High Maltose Corn Syrup | 15.64 |
| 2 | HFCS | 5.81 |
| 3 | Molasses | 1.02 |

Step 1: Weigh and cook all above liquid at 160° F.

| | | |
|---|---|---|
| 4 | Maltodextrin | 1.53 |
| 5 | Fructose | 03.06 |
| 6 | Salt | 0.51 |
| 7 | Dicalcium Phosphate Dihydrate | 5.00 |

Step 2: Add all dry ingredients, cook Brix to 88% (using the new one)

| | | |
|---|---|---|
| 8 | Canola Oil | 0.68 |
| 9 | Vanilla Flavor | 0.51 |
| 11 | Cranberry Flavor | 0.17 |
| 12 | Citric Acid | 0.07 |
| | Total | 34.00 |

Step 3: Add flavors, citric acid and oil, mix well and cook gently,
check Brix to 87%

| | | |
|---|---|---|
| 15 | Alginate Crisps (Batch # 18, 5981-15-05) | 32 |
| 16 | Oats | 14 |
| 17 | Whole Almond | 6 |
| 18 | Raisins | 7 |
| 19 | Sweetened Cranberry | 7 |

Step 4: Add 68 g syrup to dry ingredients, mix quickly
Step 5: Transfer the mass to a pan, roll it flat, cool down in
refrigerator for minimum 15 min
Step 6: Cut to L 3.5″, W 1.2″ and H 0.8″, then wrap

| | |
|---|---|
| Total | 100 |

Formula 5981-07-06

| # | Ingredients | % in Bar |
|---|---|---|
| 1 | High Maltose Corn Syrup | 15.64 |
| 2 | HFCS | 5.81 |
| 3 | Molasses | 1.02 |

Step 1: Weigh and cook all above liquid at 160° F.

| | | |
|---|---|---|
| 4 | Maltodextrin | 1.53 |
| 5 | Fructose | 3.06 |
| 6 | Salt | 0.51 |
| 7 | Dicalcium Phosphate Dihydrate | 5.00 |

Step 2: Add all dry ingredients, cook Brix to 88% (using the new one)

| | | |
|---|---|---|
| 8 | Canola Oil | 0.68 |
| 9 | Vanilla Flavor | 0.51 |
| 10 | Cranberry Flavor | 0.17 |
| 11 | Citric Acid | 0.07 |
| | Total | 34.00 |

Step 3: Add flavors, citric acid and oil, mix well and cook
gently, check Brix to 87%

| | | |
|---|---|---|
| 12 | Alginate Crisps (Batch # 18, 5981-15-05) | 32 |
| 13 | Oats | 14 |
| 14 | Whole Almond | 6 |
| 15 | Raisins | 7 |
| 16 | Sweetened Cranberry | 7 |

Step 4: Add 680 g syrup to dry ingredients, mix quickly
Step 5: Transfer the mass to a pan, roll it flat, cool down in
refrigerator for minimum 15 min
Step 6: Cut to L 3.5″, W 1.2″ and H 0.8″, then wrap

| | |
|---|---|
| Total | 100.00 |

Formula 5981-07-07, Serving size: 30 g

| # | Ingredients | % in Bar |
|---|---|---|
| 1 | High Maltose Corn Syrup | 15.64 |
| 2 | HFCS | 5.81 |
| 3 | Molasses | 1.02 |

Step 1: Weigh and cook all above liquid at 160° F.

| | | |
|---|---|---|
| 4 | Maltodextrin | 1.53 |
| 5 | Fructose | 3.06 |
| 6 | Salt | 0.51 |
| 7 | Dicalcium Phosphate Dihydrate | 5.00 |

Step 2: Add all dry ingredients, cook Brix to 87%

| | | |
|---|---|---|
| 8 | Canola Oil | 0.68 |
| 9 | Vanilla Flavor | 0.51 |
| 10 | Cranberry Flavor | 0.17 |
| 11 | Citric Acid | 0.07 |
| | Total | 34.00 |

Step 3: Add flavors, citric acid and oil, mix well and cook
gently, check Brix to 85%

| | | |
|---|---|---|
| 12 | Alginate Crisps (Batch # 18, 5981-15-05) | 22 |
| 13 | Alginate Crisps (Crushed) | 10 |
| 14 | Rolled Oats | 13 |
| 15 | Whole Almond | 5 |
| 16 | Raisins | 8 |
| 17 | Sweetened Cranberry | 8 |

Step 4: Add 680 g syrup to dry ingredients, mix quickly
Step 5: Transfer the mass to a pan, roll it flat, cool down in
refrigerator for minimum 15 min
Step 6: Cut to L 3.5″, W 1.375″ and H 0.8″, then wrap

| | |
|---|---|
| Total | 100.00 |

Formula 5981-07-08, Serving size: 30 g

| # | Ingredients | % in Bar |
|---|---|---|
| 1 | High Maltose Corn Syrup | 14.45 |
| 2 | HFCS | 5.81 |
| 3 | Molasses | 1.02 |

Step 1: Weigh and cook all above liquid at 160° F.

| | | |
|---|---|---|
| 4 | Fructose | 3.06 |
| 5 | Calcium Lacatate | 8.98 |

Step 2: Add all dry ingredients, cook Brix to 86%

| | | |
|---|---|---|
| 9 | Vanilla Flavor | 0.51 |
| 10 | Cranberry Flavor | 0.17 |
| | Total | 34.00 |

Step 3: Add flavors, citric acid and oil, mix well and cook
gently, check Brix to 84.5%

| | | |
|---|---|---|
| 12 | Alginate Crisps (Batch # 18, 5981-15-05) | 16 |
| 13 | Alginate Crisps (Crushed) | 16 |
| 14 | Rolled Oats | 13 |
| 15 | Whole Almond | 5 |
| 16 | Raisins | 8 |
| 17 | Sweetened Cranberry | 8 |

Step 4: Add 340 g syrup to dry ingredients, mix quickly
Step 5: Transfer the mass to a pan, roll it flat, cool down in
refrigerator for minimum 15 min
Step 6: Cut to L 3.5″, W 1.375″ and H 0.8″, then wrap

| | |
|---|---|
| Total | 100.00 |

Modified 5981-07-07

| # | Ingredients | % in Bar |
|---|---|---|
| 1 | High Maltose Corn Syrup | 19.83 |
| 2 | HFCS | 2.71 |
| 3 | Molasses | 1.07 |

Step 1: Weigh and cook all above liquid at 160° F.

| | | |
|---|---|---|
| 4 | Maltodextrin | 0.00 |
| 5 | Fructose | 3.75 |
| 6 | Sugar | 1.61 |
| 7 | Dicalcium Phosphate Dihydrate | 3.57 |
| 8 | Citric Acid | 0.07 |

Step 2: Add all dry ingredients, cook Brix to 88%

| | | |
|---|---|---|
| 9 | Canola Oil | 0.71 |
| 10 | Vanilla Flavor | 0.54 |
| 11 | Cranberry Flavor | 0.14 |
| | Total | 34.00 |

Step 3: Add flavors and oil, mix well and cook gently, check Brix to 87%

| | | |
|---|---|---|
| 12 | Test Crisps (5981-15-15) | 33.10 |
| 13 | Rolled Oats | 18 |
| 14 | Raisins | 4 |
| 15 | Cranberry Halves | 10.90 |

Step 4: Add 680 g syrup to dry ingredients, mix quickly
Step 5: Transfer the mass to a pan, roll it flat, cool down in
refrigerator for minimum 15 min
Step 6: Cut to L 4.0″, W 1.55″ and H 0.8″, then wrap

| | |
|---|---|
| Total | 100.00 |

What is claimed is:

1. An ingestible composition comprising at least one soluble anionic fiber and at least one soluble cation source, wherein the ingestible composition has an SE of between about 100% and about 250%.

2. An ingestible composition of claim 1, wherein the at least one soluble anionic fiber comprises alginate and pectin.

3. An ingestible composition of claim 2, wherein the alginate comprises an intermediate molecular weight form of alginate and a low molecular weight form of alginate.

4. An ingestible composition of claim 2, wherein total alginate to total pectin is from about 8:1 to about 1:8.

5. An ingestible composition of claim 1, wherein the cation is selected from the group consisting of calcium, magnesium, aluminum, manganese, iron, nickel, copper, zinc, strontium, barium, bismuth, chromium, vanadium, lanthanum, their salts and mixtures thereof.

6. An ingestible composition of claim 5, wherein the soluble cation source is selected from the group of cation salts consisting of formate, fumarate, acetate, propionate, butyrate, caprylate, valerate, lactate, citrate, malate, gluconate, chloride, potassium, phosphate and mixtures thereof.

7. An ingestible composition of claim 5, wherein the cation is calcium and wherein the soluble cation source is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, dicalcium phosphate anhydrous, calcium chloride, calcium acetate monohydrate, and mixtures thereof.

8. An ingestible composition of claim 1, wherein the at least one soluble anionic fibers to the at least one soluble cation source are present in the ingestible composition in a fiber to cation weight ratio of from about 20:1 to about 7:1.

9. An ingestible composition comprising:
i. a solid phase comprising at least one soluble anionic fiber in a total amount of from about 0.5 g to about 10 g per serving and
ii. a fluid phase in intimate contact with the solid phase, the fluid phase comprising a soluble calcium source in an amount of from about 50 to about 300 mg of elemental calcium per serving, wherein the ingestible composition has an SE of between about 100% and about 250%.

10. An ingestible composition of claim 9, wherein the at least one soluble anionic fiber comprises alginate and pectin.

11. An ingestible composition of claim 10, wherein the fluid phase is selected from the group consisting of jam, jelly, pudding, custard, frosting, icing, and coating material.

12. An ingestible composition of claim 10, wherein the fluid phase is within the solid phase.

13. An ingestible composition of claim 10, wherein the fluid phase is deposited on the solid phase.

14. An ingestible composition of claim 10, wherein the alginate comprises an intermediate molecular weight form of alginate and a low molecular weight form of alginate.

15. An ingestible composition of claim 10, wherein total alginate to total pectin is from about 8:1 to about 1:8.

16. An ingestible composition of claim 9, wherein the soluble calcium source is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, dicalcium phosphate anhydrous, calcium chloride, calcium acetate monohydrate, and mixtures thereof.

17. An ingestible composition of claim 10, wherein a weight ratio of the alginate and pectin to the soluble calcium source in the ingestible composition is from about 20:1 to about 7:1.

18. A method for inducing satiety in an animal, the method comprising the step of orally administering to the animal a serving of a preload composition, the preload composition comprising at least one soluble anionic fiber and at least one soluble cation source, wherein the preload composition has an SE of between about 100% and about 250%.

19. A method for inducing satiety in an animal, the method comprising the step of orally administering to the animal a serving of a preload composition, the preload comprising:
i. a solid phase comprising at least one soluble anionic fiber in a total amount of from about 0.5 g to about 10 g per serving and
ii. a fluid phase in intimate contact with the solid phase, the fluid phase comprising a soluble calcium source in an amount of from about 50 to about 500 mg of elemental calcium per serving, wherein the preload has an SE of between about 100% and about 250%.

20. A method for reducing caloric intake in an animal, the method comprising the step of orally administering to the animal at least one serving of a preload composition, the preload composition comprising at least one soluble anionic fibers and at least one soluble cation source, wherein the preload has an SE of between about 100% and about 250%.

21. A method for reducing caloric intake in an animal, the method comprising the step of orally administering to the animal a serving of a preload composition, the preload composition comprising:
i. a solid phase comprising at least one soluble anionic fiber in a total amount of from about 0.5 g to about 10 g per serving and
ii. a fluid phase in intimate contact with the solid phase, the fluid phase comprising a soluble calcium source in an amount of from about 50 to about 300 mg of elemental calcium per serving, wherein the preload composition has an SE of between about 100% and about 250%.

22. A method for reducing weight in an animal, the method comprising the step of orally administering to the animal a serving of an ingestible composition comprising an extruded food product, wherein the extruded food product comprises at least one soluble soluble anionic fiber and at least one soluble cation source.

23. A method for reducing weight in an animal, the method comprising the step of orally administering to the animal a serving of an ingestible composition comprising:
i. a solid phase comprising at least one soluble anionic fiber in a total amount of from about 0.5 g to about 10 g per serving and
ii. a fluid phase in intimate contact with the solid phase, the fluid phase comprising a soluble calcium source in an amount in an amount to provide from about 50 to about 300 mg of elemental calcium per serving.

24. An ingestible composition comprising:
i. a solid phase comprising at least one soluble anionic fiber in a total amount of from about 0.5 g to about 10 g per serving and
ii. a fluid phase in intimate contact with the solid phase, the fluid phase comprising a soluble calcium source in an amount to provide from about 50 to about 300 mg of elemental calcium per serving, wherein ingestible composition has an SE of between about 100% and about 250%.

25. An ingestible composition of claim 24, wherein the fluid phase is selected from the group consisting of jam, jelly, pudding, custard, frosting, icing, and enrobing material.

26. An ingestible composition of claim 24, wherein the fluid phase is within the solid phase.

27. An ingestible composition of claim 24, wherein the fluid phase is deposited on the solid phase.

28. An ingestible composition of claim 24, wherein the at least one soluble anionic fiber comprises alginate and pectin.

29. An ingestible composition of claim 28, wherein the alginate comprises an intermediate molecular weight form of alginate and a low molecular weight form of alginate.

30. An ingestible composition of claim 28, wherein a ratio of total alginate to total pectin is from about 8:1 to about 1:8.

31. An ingestible composition of claim 28, wherein the cation is calcium and wherein the source is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, dicalcium phosphate anhydrous, calcium chloride, calcium acetate monohydrate, and mixtures thereof.

32. An ingestible composition of claim 28, wherein a ratio of the alginate and pectin to the elemental calcium in the ingestible composition is from about 20:1 to about 7:1.
